(19) United States
(12) Patent Application Publication
(10) Pub. No.: US 2012/0046317 A1
(43) Pub. Date: Feb. 23, 2012

(54) NOVEL HETEROCYCLIC COMPOUNDS

(76) Inventors: Chaitanya Dutt, Gandhinagar (IN); Vijay Chauthaiwale, Gandhinagar (IN); Ramesh Chandra Gupta, Gandhinagar (IN); Manish Patel, Gandhinagar (IN); Jignesh Kotecha, Gandhinagar (IN); Sachin Latad, Gandhinagar (IN); Jaya Abraham, Gandhinagar (IN); Sanjay Srivastava, Gandhinagar (IN); Milin Rode, Gandhinagar (IN); Shailesh Deshpande, Gandhinagar (IN); Anita Chaudhary, Gandhinagar (IN); Amit Kumar Kesarwani, Gandhinagar (IN)

(21) Appl. No.: 13/318,740

(22) PCT Filed: May 6, 2010
(86) PCT No.: PCT/IN10/00295

§ 371 Date: Nov. 3, 2011

(30) Foreign Application Priority Data
May 7, 2009 (IN) 1207/MUM/2009

(51) Int. Cl. A61K 31/4535; C07D 213/54; C07D 211/34; C07D 409/02; A61K 31/4436; A61K 31/444; A61K 31/4439; A61K 31/435; A61K 31/445; A61P 25/00; A61P 9/12; A61P 9/04; A61P 27/02; A61P 9/10; A61P 25/28; A61P 13/12; A61P 17/00; C07D 409/14

(52) U.S. Cl. 514/326; 546/280.4; 546/256; 546/275.4; 546/324; 546/245; 546/212; 514/336; 514/333; 514/341; 514/355; 514/315

(57) ABSTRACT

The present invention relates to a new class of heterocyclic compounds and pharmaceutically acceptable salts thereof, process for preparing the same, pharmaceutical composition containing these compounds and to their use in treatment of diseases caused due to formation and accumulation of AGEs (Advanced Glycation end products). The compounds of the present invention are useful for the treatment of diabetic and aging-related complications caused by formation and accumulation of AGEs, such as neuropathy, nephropathy, microangiopathy, retinopathy, hypertension, heart failure, atherosclerosis, Alzheimer's disease & dermatological disorders.

### FIELD OF THE INVENTION:

The present invention relates to a new class of heterocyclic compounds and pharmaceutically acceptable salts thereof, process for preparing the same, pharmaceutical composition containing these compounds and to their use in treatment of diseases caused due to formation and accumulation of AGEs (Advanced Glycation endproducts). The compounds of the present invention are useful for the treatment of diabetic and aging-related complications caused by formation and accumulation of AGEs, such as neuropathy, nephropathy, microangiopathy, retinopathy, hypertension, heart failure, atherosclerosis, Alzheimer's disease & dermatological disorders.

### BACKGROUND OF THE INVENTION:

Maillard in 1912 found that reducing sugars, such as glucose and ribose react with proteins to form brown pigments. Further studies have shown that this is an irreversible non-enzymatic reaction, which occurs in several natural systems including stored foodstuff. Maillard reaction occurs in two stages, early and advanced. Initially, proteins react with glucose to form stable Amadori products, which subsequently cross links to form advanced glycation end products (AGEs). In most cases, the formation of AGEs also accompanies browning of the proteins and increase in the fluorescence.

Excessive accumulation of AGEs on tissue proteins has been implicated in the pathogenesis of many of the sequelae of diabetes and normal ageing (Bucala R et al. Proc. Natl. Acad. Sci., 1993, 90, 6434-6438). The formation of AGEs on long-lived connective tissue and matrix components accounts largely for the increase in collagen crosslinking that accompanies normal ageing and which occurs at an accelerated rate in diabetes. AGEs can activate cellular receptors and initiate a variety of pathophysiological response (Vasan et al. Nature 1996; 382: 275-278).

Particularly in diabetic patients, where blood glucose level is significantly higher than normal, the reaction of glucose with several proteins such as hemoglobin, lens crystallin and collagen, gives rise to the formation of AGEs, which in turn, is responsible for the macrovascular and microvascular complications associated with diabetes mellitus, such as nephropathy, microangiopathy, neuropathy, retinopathy and endothelial dysfunction. In addition, the activity of several growth factors, such as basic fibroblast growth factor, is also impaired. AGE products, unlike normal proteins in tissue, have a slower rate of turnover and replenishment. It has been reported that AGE products may in fact elicit a complex immunological reaction involving RAGE (Receptor for Advanced Glycation End Products) receptors and activation of several incompletely defined immunological processes.

Circulating advanced glycation end-products (AGEs) also bind lipoproteins and delay their clearance. As uptake via scavenger receptors is not inhibited, glycation increases the proportion of lipoproteins that are taken up via inflammatory cells and decreases the proportion taken up by hepatocytes via classical LDL receptors. This promotes the formation of atheromatous plaques and stimulates inflammation. Hyperglycemia increases the formation of oxidized LDL and glycated LDL, which are important modulators of atherosclerosis and cardiovascular death (Veiraiah A. Angiology. 2005 July-August; 56(4):431-8). Advanced glycation also may contribute to the age-related development of atherosclerosis in the general population (Bucala R. et al. 1994, Proc Natl Acad Sci, 91, 9441-9445).

The correlation between the formation and accumulation of AGEs with various diseases has been described in various literatures. Due to the clinical significance of AGEs formation, many approaches are being used to diagnose, prevent, or break AGEs formation in the body.

The formation of AGEs could be inhibited by reacting with an early glycosylation product that results from the original reaction between the target protein and glucose. The inhibition was believed to take place as the reaction between the inhibitor and the early glycosylation products appeared to interrupt the subsequent reaction of the glycosylated protein with additional protein material to form the cross linked late stage product. Known compound like aminoguanidine act to inhibit AGEs formation by such mechanism.

Several successful therapeutic approaches have also been achieved based upon blocking the accumulation of AGEs in vivo. One approach, exemplified in U.S. Pat. No. 4,758,583 concerns the inhibition of the formation of AGEs from its precursors, by the administration of agents such as aminoguanidine and related compounds.

The formation of AGEs on long-lived proteins is also associated with crosslinking of these proteins. The AGEs derived protein cross-links have been shown to be cleaved by compounds like N-phenyl thiazolium bromide (PTB), which reacts with and cleaves covalent, AGEs derived protein cross links (Vasan et al. Nature 1996; 382:275-278; U.S. Pat. No. 5,853,703). The mechanism of reducing the AGEs content in tissues is expected to take place relatively rapidly, in contrast to aminoguanidine, which acts slowly by its very nature of mechanism of action.

In a pharmacological approach to controlling levels of AGEs in tissues, especially in those tissues in which AGEs has already accumulated to levels which are responsible for sub-clinical or clinical pathology, administration of agents that reverse or break AGEs has proven successful. As described in U.S. Pat. No. 5,656,261 & U.S. Pat. No. 5,853,703, agents and methods are disclosed which reverse (or cleave or break) AGEs formation in vitro and in vivo.

TRC4149, a novel AGE breaker, by virtue of reducing AGE load found to be preserved endothelial and cardiac function in diabetic spontaneously hypertensive rats (Pathak P. et al, Eur Jr of Med res; 2008; 13;388-398). TRC4186, an experimental AGE breaker, when studied for its effect on diabetic cardiomyopathy and nephropathy in Ob-ZSF1 animal model, preserved cardiac function and reduced the severity of renal dysfunction. (Joshi D et al., J Cardiovasc Pharmacol; 2009; 54(1); 72-81). Another AGE breaker Alagabrium, has been found effective in two subsequent phase 2 clinical studies, one addressing diastolic heart failure and the other addressing systolic hypertension. Alagebrium was effective in improving cardiac function and uncontrolled systolic blood pressure, particularly in more severely affected patients (Bakris G L et al. Am J Hypertens; 2004; 17, 23S-30S).

Thus, the compounds which inhibit the accumulation of AGEs and/or break the performed AGEs can be of prime importance in therapeutic applications.

Currently, it is believed that inhibiting AGEs formation, or the breaking of existing AGEs, would be beneficial in a variety of diseases, including neuropathy, retinopathy, nephropathy, microangiopathy, hypertension, heart failure, atherosclerosis, Alzheimer's disease & dermatological disorders.

The correlation between the formation of Advanced Glycation End products (AGEs) and nephropathy is well established by several research publications. Beisswenger P J et al. (Diabetes; 1995; 44(7): 824-29) has shown that AGEs concentration in human diabetic subjects correlates with early manifestation of renal diseases. Makita et al. (N Engl J Med. 1991; 325(12): 836-42) has shown that increase in AGE peptides parallels with the severity of renal dysfunction. The above citations clearly show that advanced glycation endproducts are the principal cause of diabetic nephropathy.

Advanced glycation end products are also shown to induce expression of vascular endothelial growth factor in retinal muller cells and therefore may promote intraocular neovascularization in diabetic retinopathy (Hirata C et al., Biochem Biophys Res Commun. 1997; 236(3):712-5).

Studies have demonstrated positive effects of agents that break AGEs, such as in studies on cardiovascular complications related to aging, a condition which is accelerated in experimental diabetic conditions (Wolffenbuttel et al., Proc Natl Acad Sci 1998; 95(8):4630-4).

Advanced glycation endproducts (AGEs) have been proposed as factors involved in the development and progression of chronic heart failure (CHF). Cross-linking by advanced glycation end products results in vascular and myocardial stiffening, which are hallmarks in the pathogenesis of CHF. Additionally, stimulation of receptors by AGEs may affect endothelial function and myocardial calcium uptake and may perpetuate coronary sclerosis in CHF (Smith A J et al. Ann N Y Acad Sci. 2008; 1126; 225-30).

Advanced glycation has also been implicated in the pathology of Alzheimer's disease (Vitek M P et al., Proc Natl Acad Sci 1994; 91(11): 4766-70).

Thus, compounds which block AGEs formation, or break AGEs, can be useful for the treatment of AGE-related disorders, such as neuropathy, retinopathy, nephropathy, microangiopathy, hypertension, heart failure, atherosclerosis, Alzheimer's disease & dermatological disorders.

All patents, patent applications, and literature references cited in PCT publication WO 01/25208 & WO 02/085897 are hereby incorporated by reference in their entirety. In the case of inconsistencies, the present disclosure, including definitions, will prevail. The said applications i.e. WO 01/25208 & WO 02/085897 disclose the substituted pyridinium derivative including quarternized derivative and pharmaceutically acceptable salt thereof, which are useful in the treatment of diseases caused due to formation and accumulation of AGEs (Advanced Glycation Endproducts).

However, it has been observed that the pyridinium derivatives as disclosed in WO 01/25208 & WO 02/085897 require high drug loading to attain required blood level concentration and/or to exhibit desired exposure at its target site.

To overcome such type of the problems there are various approaches known in the arts. Among them, prodrug strategies or methodologies is known to markedly enhance properties of a drug or to overcome an inherent deficiency in the pharmaceutical or pharmacokinetic properties of a drug. A variety of prodrug strategies exist which provide choices in modulating the conditions for regeneration of the parent drug such as salt formation, ester formation, conjugate or complex formation, amide formation, etc.

Surprisingly, the inventors of the present invention have found that reduced compounds of present disclosure, when administered, achieve desired blood concentration and/or exposure at its target tissue site of its pyridinium derivatives with significantly lower drug loading in comparison of pyridinium derivatives as disclosed in WO 01/25208 & WO 02/085897.

### SUMMARY OF THE INVENTION

The first embodiment of the present invention is to provide a new class of heterocyclic compounds and pharmaceutically acceptable salt thereof, which are useful for the management of diabetic and aging related complications and particularly in the treatment of complications of diabetes mellitus such as neuropathy, nephropathy, microangiopathy, hypertension, heart failure, retinopathy, atherosclerosis, Alzheimer's disease & dermatological disorders.

Another embodiment of the present invention is to provide new class of heterocyclic compounds and pharmaceutically acceptable salt thereof, which exhibit AGE breaking and/or inhibiting activities.

Yet another embodiment of the present invention is to provide a method of preparation of new class of heterocyclic compounds and pharmaceutically acceptable salt thereof, which exhibit AGE breaking and/or inhibiting activities.

Yet another embodiment of the present invention is to provide pharmaceutical compositions of a new class of heterocyclic compounds and pharmaceutically acceptable salt thereof and one or more of pharmaceutically acceptable excipient(s) or other media normally employed in preparing such compositions.

Yet another embodiment of the present invention is to provide pharmaceutical compositions of a new class of heterocyclic compounds, wherein the composition is acid resistant composition.

Yet another embodiment of the present invention is to provide a method of treating a disease condition caused by formation and accumulation of AGEs by administration of therapeutically effective amount of a compound of the present invention to a mammal in need thereof.

Yet another embodiment of the present invention is to provide a method of treating a neuropathy, nephropathy, microangiopathy, hypertension, heart failure, retinopathy, atherosclerosis, Alzheimer's disease & dermatological disorders by administration of therapeutically effective amount of a compound of the present invention to a mammal in need thereof.

Yet another embodiment of the present invention is to provide a method of treating a disease conditions caused by formation and accumulation of AGEs by administration of therapeutically effective amount of a compound of the invention to a mammal in need thereof in combination with one or more other AGE breakers/inhibitors and/or anti-diabetic agents.

Yet another embodiment of the present invention is use of a compound of the invention for the manufacture of medicament for treatment of a disease conditions caused by formation and accumulation of AGEs.

Yet another embodiment of the present invention is use of a compound of the invention for the manufacture of medicament for treatment of neuropathy, nephropathy, microangiopathy, hypertension, heart failure, retinopathy, atherosclerosis, Alzheimer's disease & dermatological disorders caused by formation and accumulation of AGEs.

Yet another embodiment of the present invention is use of a compound of the invention for the manufacture of medicament for treatment of a disease conditions caused by formation and accumulation of AGEs in combination with one or more other AGE breakers/inhibitors and/or anti-diabetic agents.

Yet another embodiment of the present invention is a compound of the invention adapted for treatment of neuropathy, nephropathy, microangiopathy, hypertension, heart failure, retinopathy, atherosclerosis, Alzheimer's disease & dermatological disorders caused by formation and accumulation of AGEs by administration of therapeutically effective amount of the said compound.

### DESCRIPTION OF THE FIGURES

FIG. 1 depicts a graph showing the single dose pharmacokinetics of Reference compound-T and compound no. 100 in Wistar rats.

FIG. 2 depicts a graph showing the single dose pharmacokinetics of Reference compound-T and Compound no. 100 in Dogs.

### DETAILED DESCRIPTION

In one embodiment, the present invention provides a new class of heterocyclic compound of formula (I) and pharmaceutically acceptable salt thereof,

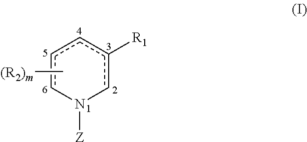

Wherein, the dotted line in nitrogen containing ring represents:
(a) two double bond between either (i) at C2-C3 and C5-C6, or (ii) at C2-C3 and C4-C5, or (iii) at C3-C4 and C5-C6, or
(b) one double between either (i) at C2-C3 or (ii) at C3-C4 or (iii) at C4-C5 or (iv) at C5-C6, or
(c) absence of double bond i.e. a saturated ring system;

R₁ is —COR₃ or 5 membered heterocyclic ring having the following formula;

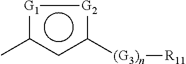

G₁ & G₂ are independently N, NH, NR₁₂, S or O to form heterocyclic ring system, which may also be either partially or fully saturated;

G₃ is —(C₁-C₁₂)alkylene-P or —(C₁-C₁₂)alkylene, wherein P is sulfur, oxygen or nitrogen, and n is 0 or 1;

Z is i) —CH₂—C(O)—Rₓ or ii) R_{y};

Rₓ is R₇, OR₇, —N(R₇)(R₁₀), —N═C(R₇)(R₁₀), —N(R₇)N(R₇)(R₁₀), —N(R₇)N═C(R₇)(R₁₀), —CH(R₇)C(O)R₈ or a compound having one of the following formula

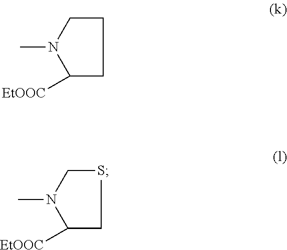

R_{y} is selected from the group consisting of hydrogen, linear or branched (C₁-C₁₂)alkyl, (C₂-C₁₂)alkenyl, (C₃-C₇)cycloalkyl, (C₅-C₇)cycloalkenyl, bicycloalkyl, CH₂(CO)R₁₃, CH₂(CO)NHR₁₄, CH₂(CO)NR₁₄R₁₅ and CH₂(CO)OR₁₃;

R₂ at each occurrence is halogen, OR₇, NO₂, alkyl, aryl, heterocyclyl, formyl, oxo, —NR₇R₁₀, —N═C(R₇)(R₁₀), —SR₇, —SO₂NH₂, —SO₂ alkyl, —SO₂ aryl, N═C(R₁₄)(R₁₅), —NR₁₄R₁₅, —OR₁₄, perhaloalkyl, —O(CO)R₁₄, —NH(CO)R₁₄, (C₂-C₁₂)alkenyl, (C₃-C₇)cycloalkyl, (C₅-C₇)cycloalkenyl, bicycloalkyl, bicycloalkenyl, heterocycloalkyl, or aralkyl;

m is 0, 1, 2 or 3;

R₃ is —R₄—R₅, —N(R₇)N(R₇)R₉ or a compound having one of the following formula

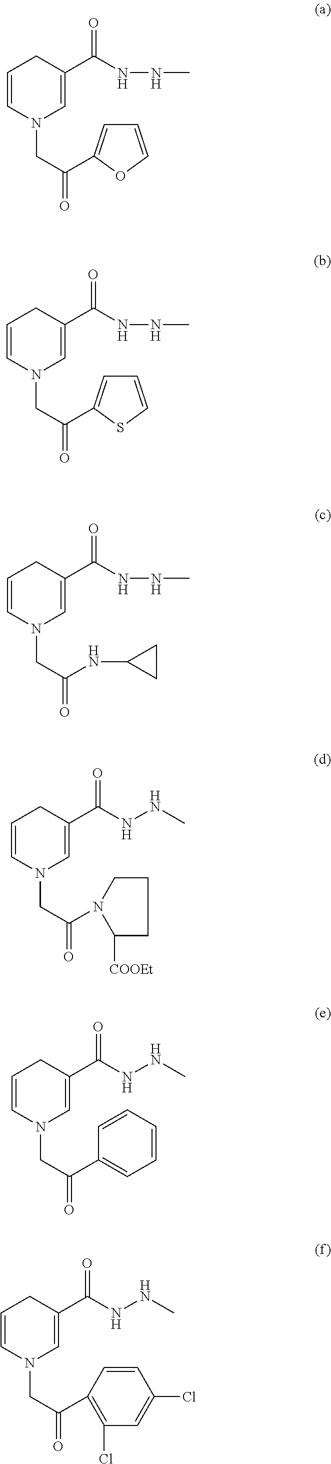

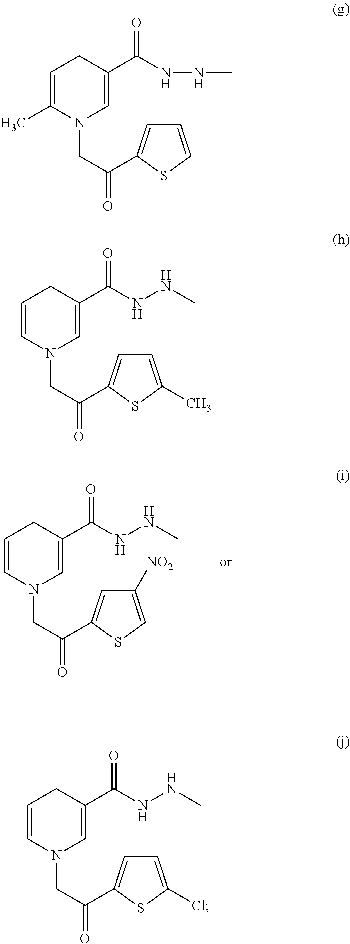

R₄ is —N(R₇)R₆O—, —N(R₇)R₆N(R₇)—, —OR₆O or —OR₆N(R₇)—, where R₆ is alkylene;

R₅ is hydrogen, alkyl, aryl, heterocyclyl, —COR₇, SO₂R₇, —C(S)NHR₇, —C(═NH)NHR₇, —COR₁₀, —C(O)NHR₇

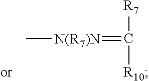

where R₇ is H, alkyl, aryl or heterocyclyl;

R₈ is R₇, OR₇ or NR₇R₁₀;

R₉ is selected from the group consisting of hydrogen, alkyl, aryl, heterocyclyl, C(O)R₁₀, —SO₂R₁₀, —C(S)NHR₁₀, —C(═NH)NH(R₁₀) and —C(O)NHR₁₀;

R₁₀ is selected from the group consisting of H, alkyl, alkoxy, aryl and heterocyclyl;

R₁₁ is selected from the group consisting hydrogen, linear or branched (C₁-C₁₂)alkyl, (C₂-C₁₂)alkenyl, (C₃-C₇)cycloalkyl, (C₅-C₇)cycloalkenyl, bicycloalkyl, bicycloalkenyl, heterocycloalkyl, aryl, aralkyl, heterocyclyl and compound (m),

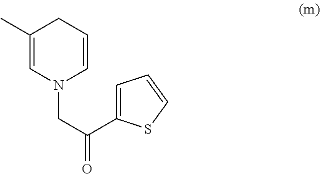

wherein in R11 one or more heteroatoms when present are independently O, N, or S and is optionally substituted, wherein the substituents are selected from a first group consisting of halogen, hydroxy, nitro, cyano, amino, oxo and oxime or from a second group consisting of linear or branched (C₁-C₈)alkyl, (C₃-C₇)cycloalkyl, alkylcycloalkyl, perhaloalkyl, perhalocycloalkyl, aryl, aralkyl, alkylaryl, aralkoxylalkyl, perhaloaryl, alkylheterocycloalkyl, heterocyclyloalkyl, perhaloheterocyclyloalkyl, heterocyclyl, perhaloheteroaryl, alkoxyalkyl, thioalkyl and thioaryl, wherein the substitutents from said second group are optionally substituted by halogen, hydroxy, nitro, cyano, amino, oxo, perhaloalkyl (C₁-C₆) and oxime and are optionally and independently bridged by —CO, —(CO)O—, —(CO)NH—, —NH—, —NR₁₄—, —O—, —S—, —(SO)—, —(SO₂), —(SO₂)NH—, or —NH(CO)—;

R₁₂ and R₁₃ are independently selected from the group consisting of linear or branched (C₁-C₈)alkyl, (C₃-C₇)cycloalkyl, alkylcycloalkyl, perhaloalkyl, perhalocycloalkyl, aryl, aralkyl, alkylaryl, aralkoxylalkyl, perhaloaryl, alkylheterocycloalkyl, heterocycloalkyl, perhaloheterocycloalkyl, heterocyclyl, perhaloheterocyclyl, —COalkyl, —COaryl, benzoyl, alkoxyalkyl, thioalkyl and thioaryl wherein members of said group are optionally substituted by R₁₆;

R₁₄ and R₁₅ are independently selected from the group consisting of linear or branched (C₁-C₁₂)alkyl, alkoxyaryl, alkoxyalkyl, alkoxycycloalkyl, alkoxyaryl, perhaloalkyl, (C₂-C₁₂)alkenyl, (C₃-C₇)cycloalkyl, perhalocycloalkyl, haloheterocycloalkyl, cyanoheterocycloalkyl, perhaloheterocycloalkyl, (C₅-C₇)cycloalkenyl, bicycloalkyl, bicycloalkenyl, heterocycloalkyl, aryl, aralkyl, heterocyclyl, perhaloaryl and perhaloheterocyclyl wherein substituents of said group are optionally substituted by R₁₆;

R₁₆ is selected from halogen, hydroxy, nitro, cyano, amino, oxo, perhaloalkyl (C₁-C₆), or oxime;

with the proviso that

(i) when R₁ is —C(O)R₃, then Z is —CH₂—C(O)-Rx;

(ii) when Z is —CH₂—C(O)—Rₓ and Rₓ is OR₇ then R₇ is not hydrogen.

A family of specific compound of particular interest from the new class of heterocyclic compound of the present invention and pharmaceutically acceptable salts thereof is as follows:

5-bromo-N′-(methylsulfonyl)-1-[2-oxo-2-(thiophen-2-yl)ethyl]-1,4-dihydropyridine-3-carbohydrazide (Compound no. 1);

ethyl 2-({1-[2-oxo-2-(thiophen-2-yl)ethyl]-1,4-dihydropyridin-3-yl}carbonyl)hydrazinecarboxylate (Compound no. 2);

2-[4-{[(3-{1-[2-oxo-2-(thiophen-2-yl)ethyl]-1,4-dihydropyridin-3-yl}-1H-pyrazol-5- yl)methyl]sulfanyl}pyridin-1(4H)-yl]-1-(thiophen-2-yl)ethanone (Compound no. 3);

2-[3-{5-[(3,5-dimethyl-1H-pyrazol-1-yl)methyl]-1H-pyrazol-3-yl}pyridin-1(4H)-yl]-1-(thiophen-2-yl)ethanone (Compound no. 4);

2-{3-[5-benzyl-1-(pyridin-2-yl)-1H-pyrazol-3-yl]pyridin-1(4H)-yl}-1-(thiophen-2-yl)ethanone (Compound no. 5);

1-[2-(5-chlorothiophen-2-yl)-2-oxoethyl]-N′-(methylsulfonyl)-1,4-dihydropyridine-3-carbohydrazide (Compound no. 6);

1-[2-(4-nitrothiophen-2-yl)-2-oxoethyl]-N′-({1-[2-(4-nitrothiophen-2-yl)-2-oxoethyl]-1,4-dihydropyridin-3-yl}carbonyl)-1,4-dihydropyridine-3-carbohydrazide (Compound no. 7);

6-methyl-N′-(methylsulfonyl)-1-[2-oxo-2-(thiophen-2-yl)ethyl]-1,4-dihydropyridine-3-carbohydrazide (Compound no. 8);

1-[2-(5-methylthiophen-2-yl)-2-oxoethyl]-N′-({1-[2-(5-methylthiophen-2-yl)-2-oxoethyl]-1,4-dihydropyridin-3-yl}carbonyl)-1,4-dihydropyridine-3-carbohydrazide (Compound no. 9);

diethyl 1,1′-{hydrazine-1,2-diylbis[carbonylpyridine-3,1(4H)-diyl(1-oxoethane-2,1-diyl)]}dipyrrolidine-2-carboxylate (Compound no. 10);

ethyl 3-{[3-{[2-(methylsulfonyl)hydrazinyl]carbonyl}pyridin-1(4H)-yl]acetyl}-1,3-thiazolidine-4-carboxylate (Compound no. 11);

1-[2-(5-chlorothiophen-2-yl)-2-oxoethyl]-N′-({1-[2-(5-chlorothiophen-2-yl)-2-oxoethyl]-1,4-dihydropyridin-3-yl}carbonyl)-1,4-dihydropyridine-3-carbohydrazide (Compound no. 12);

N′-(methylsulfonyl)-1-[2-(5-methylthiophen-2-yl)-2-oxoethyl]-1,4-dihydropyridine-3-carbohydrazide (Compound no. 13);

N′-(methylsulfonyl)-1-[2-(4-nitrothiophen-2-yl)-2-oxoethyl]-1,4-dihydropyridine-3-carbohydrazide (Compound no. 14);

N-phenyl-2-{3-[(2-phenylhydrazinyl)carbonyl]pyridin-1(4H)-yl}acetamide (Compound no. 15);

2-[({1-[2-oxo-2-(phenylamino)ethyl]-1,4-dihydropyridin-4-yl}carbonyl)amino]ethyl benzoate (Compound no. 16);

N′-(methylsulfonyl)-1-[2-(5-nitrothiophen-2-yl)-2-oxoethyl]-1,4-dihydropyridine-3-carbohydrazide (Compound no. 17);

1-[2-oxo-2-(thiophen-2-yl)ethyl]-N′-[(trifluoromethyl)sulfonyl]-1,4-dihydropyridine-3-carbohydrazide (Compound no. 18);

1-[2-oxo-2-(thiophen-2-yl)ethyl]-N′-phenyl-1,4-dihydropyridine-3-carbohydrazide (Compound no. 19);

1-[2-(2,4-dichlorophenyl)-2-oxoethyl]-1,4-dihydropyridine-3-carboxamide (Compound no. 20);

N′-[(4-methoxyphenyl)sulfonyl]-1-[2-oxo-2-(thiophen-2-yl)ethyl]-1,4-dihydropyridine-3-carbohydrazide (Compound no. 21);

2-{[1-(2-oxo-2-phenylethyl)-1,4-dihydropyridin-3-yl]carbonyl}-N-phenylhydrazine carboxamide (Compound no. 22);

2-[3-{[2-(benzylsulfonyl)hydrazinyl]carbonyl}pyridin-1(4H)-yl]-N-phenylacetamide (Compound no. 23);

N′-(methylsulfonyl)-1-(2-oxo-2-phenylethyl)-1,4-dihydropyridine-4-carbohydrazide (Compound no. 24);

1-(2-oxo-2-phenylethyl)-N′-phenyl-1,4-dihydropyridine-3-carbohydrazide (Compound no. 25);

N′-[(4-methoxyphenyl)sulfonyl]-1-(2-oxo-2-phenylethyl)-1,4-dihydropyridine-3-carbohydrazide (Compound no. 26);

1-(2-oxo-2-phenylethyl)-1,4-dihydropyridine-3-carboxamide (Compound no. 27);

2-({[1-(2-oxo-2-phenylethyl)-1,4-dihydropyridin-4-yl]carbonyl}amino)ethyl benzoate (Compound no. 28);

N-cyclopropyl-2-[3-{5-[(3,5-dimethyl-1H-pyrazol-1-yl)methyl]-1H-pyrazol-3-yl}pyridin-1(4H)-yl]acetamide (Compound no. 29);

2-[3-{5-[(3,5-dimethyl-1H-pyrazol-1-yl)methyl]-1H-pyrazol-3-yl}pyridin-1(4H)-yl]-1-(5-nitrothiophen-2-yl)ethanone (Compound no. 30);

2-[3-{3-[(3,5-dimethyl-1H-pyrazol-1-yl)methyl]-1-(pyridin-2-yl)-1H-pyrazol-5-yl}pyridin-1(4H)-yl]-1-(thiophen-2-yl)ethanone (Compound no. 31);

2-[3-(5-benzyl-1H-pyrazol-3-yl)pyridin-1(4H)-yl]-N-cyclopropylacetamide (Compound no. 32);

2,2′-[1H-pyrazole-3,5-diylbis(pyridine-3,1(4H)-diyl)]bis[1-(thiophen-2-yl)ethanone](Compound no. 33);

2-[3-(5-benzyl-1-phenyl-1H-pyrazol-3-yl)pyridin-1(4H)-yl]-1-(thiophen-2-yl)ethanone (Compound no. 34);

2-[3-(5-benzyl-1H-pyrazol-3-yl)pyridin-1(4H)-yl]-1-(5-methylthiophen-2-yl)ethanone (Compound no. 35);

2-[3-{5-[(3,5-dimethyl-1H-pyrazol-1-yl)methyl]-1-phenyl-1H-pyrazol-3-yl}pyridin-1(4H)-yl]-1-(thiophen-2-yl)ethanone (Compound no. 36);

N′-({1-[2-oxo-2-(thiophen-2-yl)ethyl]-1,4-dihydropyridin-3-yl}carbonyl)pyridine-3-carbohydrazide (Compound no. 37);

2-[3-(5-benzyl-1H-pyrazol-3-yl)pyridin-1(4H)-yl]-1-phenylethanone (Compound no. 38);

2-[3-(5-benzyl-1-phenyl-1H-pyrazol-3-yl)pyridin-1(4H)-yl]-N-cyclopropylacetamide (Compound no. 39);

2-[3-{5-[(3,5-dimethyl-1H-pyrazol-1-yl)methyl]-1H-pyrazol-3-yl}pyridin-1(4H)-yl]-1-phenylethanone (Compound no. 40);

2-[3-{5-[(3,5-dimethyl-1H-pyrazol-1-yl)methyl]-1H-pyrazol-3-yl}pyridin-1(4H)-yl]-1-(5-methylthiophen-2-yl)ethanone (Compound no. 41);

2-[3-(5-benzyl-1-phenyl-1H-pyrazol-3-yl)pyridin-1(4H)-yl]-1-phenylethanone (Compound no. 42);

2-{3-[5-(2-cyclohexylethyl)-1H-pyrazol-3-yl]pyridin-1(4H)-yl}-1-(5-methylthiophen-2-yl)ethanone (Compound no. 43);

2-{3-[5-(2-cyclohexylethyl)-1H-pyrazol-3-yl]pyridin-1(4H)-yl}-N-cyclopropylacetamide (Compound no. 44);

2-{3-[5-(2-cyclohexylethyl)-1H-pyrazol-3-yl]pyridin-1(4H)-yl}-1-phenylethanone (Compound no. 45);

2-[3-(5-benzyl-1-cyclohexyl-1H-pyrazol-3-yl)pyridin-1(4H)-yl}-N-cyclopropylacetamide (Compound no. 46);

2-{3-[5-(phenoxymethyl)-1H-pyrazol-3-yl]pyridin-1(4H)-yl}-1-(thiophen-2-yl)ethanone (Compound no. 47);

2-[3-(5-benzyl-1H-pyrazol-3-yl)pyridin-1(4H)-yl]-N-(tricyclo[3.3.1.13,7]dec-1-yl)acetamide (Compound no. 48);

2-[3-{5-[(3,5-dimethyl-1H-pyrazol-1-yl)methyl]-1-phenyl-1H-pyrazol-3-yl}pyridin-1(4H)-yl]-1-phenylethanone (Compound no. 49);

2-[3-{1-cyclohexyl-5-[(3,5-dimethyl-1H-pyrazol-1-yl)methyl]-1H-pyrazol-3-yl}pyridin-1(4H)-yl]-1-(4-nitrothiophen-2-yl)ethanone (Compound no. 50);

2-{3-[3-(2-cyclohexylethyl)-1H-pyrazol-5-yl]pyridin-1(4H)-yl}-1-(4-nitrothiophen-2-yl)ethanone (Compound no. 51);

2-{3-[3-(phenoxymethyl)-1-phenyl-1H-pyrazol-5-yl]pyridin-1(4H)-yl}-1-(thiophen-2-yl)ethanone (Compound no. 52);

2-[3-(3-benzyl-1-phenyl-1H-pyrazol-5-yl)pyridin-1(4H)-yl]-1-(4-nitrothiophen-2-yl)ethanone (Compound no. 53);

N-cyclopropyl-2-{3-[3-(phenoxymethyl)-1H-pyrazol-5-yl]pyridin-1(4H)-yl}acetamide (Compound no. 54);

1-phenyl-2-[3-(phenylcarbonyl)pyridin-1(4H)-yl]ethanone (Compound no. 55);

2-[3-{1-cyclohexyl-3-[(3,5-dimethyl-1H-pyrazol-1-yl)methyl]-1H-pyrazol-5-yl}pyridin-1(4H)-yl]-N-cyclopropylacetamide (Compound no. 56);

1-(5-chlorothiophen-2-yl)-2-{3-[3-(phenoxymethyl)-1H-pyrazol-5-yl]pyridin-1(4H)-yl}ethanone (Compound no. 57);

2-{3-[3-(phenoxymethyl)-1-phenyl-1H-pyrazol-5-yl]pyridin-1(4H)-yl}-1-phenylethanone (Compound no. 58);

2-[3-{1-cyclohexyl-3-[(3,5-dimethyl-1H-pyrazol-1-yl)methyl]-1H-pyrazol-5-yl}pyridin-1(4H)-yl]-1-(thiophen-2-yl)ethanone (Compound no. 59);

N-cyclopropyl-2-{3-[3-(phenoxymethyl)-1-phenyl-1H-pyrazol-5-yl]pyridin-1(4H)-yl}acetamide (Compound no. 60);

2-{3-[3-(2-cyclohexylethyl)-1-phenyl-1H-pyrazol-5-yl]pyridin-1(4H)-yl}-1-(thiophen-2-yl)ethanone (Compound no. 61);

2-{3-[1-cyclohexyl-3-(phenoxymethyl)-1H-pyrazol-5-yl]pyridin-1(4H)-yl}-1-(thiophen-2-yl)ethanone (Compound no. 62);

1-[2-(2,5-dichlorophenyl)-2-oxoethyl]-N-(5-methyl-4,5,6,7-tetrahydro-1,3-benzothiazol-2-yl)-1,4-dihydropyridine-3-carboxamide (Compound no. 63);

1-(naphthalen-2-yl)-2-{3-[3-(phenoxymethyl)-1H-pyrazol-5-yl]pyridin-1(4H)-yl}ethanone (Compound no. 64);

1-benzyl-3-(3-benzyl-1H-pyrazol-5-yl)-1,4-dihydropyridine (Compound no. 65);

2-{3-[3-(naphthalen-1-ylmethyl)-1H-pyrazol-5-yl]pyridin-1(4H)-yl}-1-(thiophen-2-yl)ethanone (Compound no. 66);

1-phenyl-2-{3-[3-(thiophen-2-ylmethyl)-1H-pyrazol-5-yl]pyridin-1(4H)-yl}ethanone (Compound no. 67);

1-(5-methylthiophen-2-yl)-2-{3-[3-(2-phenylethyl)-1H-pyrazol-5-yl]pyridin-1(4H)-yl}ethanone (Compound no. 68);

1-(5-methylthiophen-2-yl)-2-{3-[3-(3-phenoxypropyl)-1H-pyrazol-5-yl]pyridin-1(4H)-yl}ethanone (Compound no. 69);

3-(3-benzyl-1H-pyrazol-5-yl)-1-(propan-2-yl)-1,4-dihydropyridine (Compound no. 70);

1-(5-methylthiophen-2-yl)-2-[3-{3-[(phenylsulfanyl)methyl]-1H-pyrazol-5-yl}pyridin-1(4H)-yl]ethanone (Compound no. 71);

N-(2-hydroxyethyl)-1-(2-oxo-2-phenylethyl)-1,4-dihydropyridine-3-carboxamide (Compound no. 72);

2-[3-{3-[(1-methyl-1H-indol-3-yl)methyl]-1H-1-pyrazol-5-yl}pyridin-1(4H)-yl]-1-(thiophen-2-yl)ethanone (Compound no. 73);

2-[3-(3-methyl-1H-pyrazol-5-yl)pyridin-1(4H)-yl]-1-(naphthalen-2-yl)ethanone (Compound no. 74);

2-[3-(3-benzyl-1H-pyrazol-5-yl)pyridin-1(4H)-yl]-N-(2,3-dihydro-1,4-benzodioxin-6-yl)acetamide (Compound no. 75);

2-[3-bromo-5-(3-phenyl-1H-pyrazol-5-yl)pyridin-1(4H)-yl]-1-(thiophen-2-yl)ethanone (Compound no. 76);

2-[3-(3-phenyl-1H-pyrazol-5-yl)pyridin-1(4H)-yl]-1-(thiophen-2-yl)ethanone (Compound no. 77);

1-[2-oxo-2-(thiophen-2-yl)ethyl]-1,4-dihydropyridine-3-carboxamide (Compound no. 78);

1-(2-oxo-2-phenylethyl)-N′-{[1-(2-oxo-2-phenylethyl)-1,4-dihydropyridin-3-yl]carbonyl}-1,4-dihydropyridine-3-carbohydrazide (Compound no. 79);

1-(2-oxo-2-phenylethyl)-N-(4-sulfamoylphenyl)-1,4-dihydropyridine-3-carboxamide (Compound no. 80);

1-[2-(2,4-dichlorophenyl)-2-oxoethyl]-N′-({1-[2-(2,4-dichlorophenyl)-2-oxoethyl]-1,4-dihydropyridin-3-yl}carbonyl)-1,4-dihydropyridine-3-carbohydrazide (Compound no. 81);

propan-2-yl 1-(2-oxo-2-phenylethyl)-1,4-dihydropyridine-3-carboxylate (Compound no. 82);

N-(2-hydroxyethyl)-1-(2-oxopropyl)-1,4-dihydropyridine-3-carboxamide (Compound no. 83);

N-(2-hydroxyethyl)-1-[2-oxo-2-(thiophen-2-yl)ethyl]-1,4-dihydropyridine-3-carboxamide (Compound no. 84);

1-[2-oxo-2-(thiophen-2-yl)ethyl]-N′-({1-[2-oxo-2-(thiophen-2-yl)ethyl]-1,4-dihydropyridin-3-yl}carbonyl)-1,4-dihydropyridine-3-carbohydrazide (Compound no. 85);

propan-2-yl 1-[2-(2,4-dichlorophenyl)-2-oxoethyl]-1,4-dihydropyridine-3-carboxylate (Compound no. 86);

methyl 1-(2-oxo-2-phenylethyl)-1,4-dihydropyridine-3-carboxylate (Compound no. 87);

N-(4-methyl-1,3-thiazol-2-yl)-1-(2-oxo-2-phenylethyl)-1,4-dihydropyridine-3-carboxamide (Compound no. 88);

N-butyl-1-[2-oxo-2-(phenylamino)ethyl]-1,4-dihydropyridine-3-carboxamide (Compound no. 89);

butyl 1-[2-oxo-2-(phenylamino)ethyl]-1,4-dihydropyridine-3-carboxylate (Compound no. 90);

N-(2-hydroxyethyl)-1-[2-oxo-2-(phenylamino)ethyl]-1,4-dihydropyridine-3-carboxamide (Compound no. 91);

1-(2-hydrazinyl-2-oxoethyl)-N-(2-hydroxyethyl)-1,4-dihydropyridine-3-carboxamide (Compound no. 92);

1-(2-hydrazinyl-2-oxoethyl)-1,4-dihydropyridine-3-carbohydrazide (Compound no. 93);

butyl 1-[2-(2,4-dichlorophenyl)-2-oxoethyl]-1,4-dihydropyridine-3-carboxylate (Compound no. 94);

N-butyl-1-[2-(2,4-dichlorophenyl)-2-oxoethyl]-1,4-dihydropyridine-3-carboxamide (Compound no. 95);

2-[({1-[2-(2,4-dichlorophenyl)-2-oxoethyl]-1,4-dihydropyridin-3-yl}carbonyl)amino]ethyl benzoate (Compound no. 96);

1-[2-oxo-2-(thiophen-2-yl)ethyl]-N′-(pyridin-2-yl)-1,4-dihydropyridine-3-carbohydrazide (Compound no. 97);

1-(2-oxo-2-phenylethyl)-N′-(pyridin-2-yl)-1,4-dihydropyridine-3-carbohydrazide (Compound no. 98);

1-(2-oxo-2-phenylethyl)-1,4-dihydropyridine-3-carbohydrazide (Compound no. 99);

N′-(methylsulfonyl)-1-[2-oxo-2-(thiophen-2-yl)ethyl]-1,4-dihydropyridine-3-carbohydrazide (Compound no. 100);

N′-(methylsulfonyl)-1-(2-oxo-2-phenylethyl)-1,4-dihydropyridine-3-carbohydrazide (Compound no. 101);

1-(2-oxo-2-phenylethyl)-N′-(phenylsulfonyl)-1,4-dihydropyridine-3-carbohydrazide (Compound no. 102);

6-chloro-1-(2-oxo-2-phenylethyl)-N′-(phenylsulfonyl)-1,4-dihydropyridine-3-carbohydrazide (Compound no. 103);

2-[({1-[2-oxo-2-(thiophen-2-yl)ethyl]-1,4-dihydropyridin-4-yl}carbonyl)amino]ethyl benzoate (Compound no. 104);

2-[(methoxycarbonyl)oxy]ethyl 1-(2-oxo-2-phenylethyl)-1,4-dihydropyridine-3-carboxylate (Compound no. 105);

2-methoxyethyl 1-[2-(2,4-dichlorophenyl)-2-oxoethyl]-1,4-dihydropyridine-3-carboxylate (Compound no. 106);

2-[({1-[2-oxo-2-(phenylamino)ethyl]-1,4-dihydropyridin-3-yl}carbonyl)amino]ethyl benzoate (Compound no. 107);

N-phenyl-2-[3-{[2-(phenylsulfonyl)hydrazinyl]carbonyl}pyridin-1(4H)-yl]acetamide (Compound no. 108);

2-[3-({2-[(4-methylphenyl)sulfonyl]hydrazinyl}carbonyl)pyridin-1(4H)-yl]-N-phenylacetamide (Compound no. 109);

N-phenyl-2-[4-{[2-(phenylsulfonyl)hydrazinyl]carbonyl}pyridin-1(4H)-yl]acetamide (Compound no. 110);

2-[(phenylcarbonyl)oxy]ethyl 1-(2-oxo-2-phenylethyl)-1,4-dihydropyridine-3-carboxylate (Compound no. 111);

1-[2-oxo-2-(thiophen-2-yl)ethyl]-N′-(phenylcarbonyl)-1,4-dihydropyridine-3-carbohydrazide (Compound no. 112);

N′-(benzylsulfonyl)-1-(2-oxo-2-phenylethyl)-1,4-dihydropyridine-3-carbohydrazide (Compound no. 113);

N-(2-hydroxyethyl)-1-[2-oxo-2-(phenylamino)ethyl]-1,4-dihydropyridine-3-carboxamide (Compound no. 114);

N′-(3-cyclohexylpropanoyl)-1-[2-oxo-2-(thiophen-2-yl)ethyl]-1,4-dihydropyridine-3-carbohydrazide (Compound no. 115);

2-[3-{[2-(3-cyclohexylpropanoyl)hydrazinyl]carbonyl}pyridin-1(4H)-yl]-N-phenylacetamide (Compound no. 116);

2-[({1-[2-oxo-2-(thiophen-2-yl)ethyl]-1,4-dihydropyridin-3-yl}carbonyl)amino]ethyl benzoate (Compound no. 117);

2-({[1-(4-ethoxy-2,4-dioxobutyl)-1,4-dihydropyridin-3-yl]carbonyl}amino)ethyl benzoate (Compound no. 118);

1-[2-(furan-2-yl)-2-oxoethyl]-N′-({1-[2-(furan-2-yl)-2-oxoethyl]-1,4-dihydropyridin-3-yl}carbonyl)-1,4-dihydropyridine-3-carbohydrazide (Compound no. 119);

1-[2-(2,5-dichlorophenyl)-2-oxoethyl]-N-(2-methoxyethyl)-1,4-dihydropyridine-3-carboxamide (Compound no. 120);

2,2′-[4hydrazine-1,2-diylbis(carbonylpyridine-3,1(4H)-diyl)]bis(N-cyclopropylacetamide) (Compound no. 121);

1-{1-[2-oxo-2-(thiophen-2-yl)ethyl]-1,4-dihydropyridin-3-yl}-4-phenylbutane-1,3-dione (Compound no. 122);

1-[2-(cyclopropylamino)-2-oxoethyl]-N-(2-methoxyethyl)-1,4-dihydropyridine-3-carboxamide (Compound no. 123);

2,2′-[hydrazine-1,2-diylbis(carbonylpyridine-3,1(4H)-diyl)]bis[N-(propan-2-yl)acetamide](Compound no. 124);

2-chloro-N′-({1-[2-oxo-2-(thiophen-2-yl)ethyl]-1,4-dihydropyridin-3-yl}carbonyl)pyridine-3-carbohydrazide (Compound no. 125);

2-[3-{[2-(methylsulfonyl)hydrazinyl]carbonyl}pyridin-1(4H)-yl]-N-(propan-2-yl)acetamide (Compound no. 126);

N′-(methylsulfonyl)-1-[2-oxo-2-(pyrrolidin-1-yl)ethyl]-1,4-dihydropyridine-3-carbohydrazide (Compound no. 127);

1-[1-(2-oxo-2-phenylethyl)-1,4-dihydropyridin-3-yl]-4-phenylbutane-1,3-dione (Compound no. 128);

2,2′-[4hydrazine-1,2-diylbis(carbonylpyridine-3,1(4H)-diyl)]diacetic acid (Compound no. 129);

5-bromo-N-(2-methoxyethyl)-1-[2-oxo-2-(thiophen-2-yl)ethyl]-1,4-dihydropyridine-3-carboxamide (Compound no. 130);

methyl 5-{[2-({1-[2-oxo-2-(thiophen-2-yl)ethyl]-1,4-dihydropyridin-3-yl}carbonyl)hydrazinyl]carbonyl}pyridine-2-carboxylate (Compound no. 131);

2-[3-(3-benzyl-1H-pyrazol-5-yl)pyridin-1(4H)-yl]-1-(thiophen-2-yl)ethanone (Compound no. 132);

1,3-bis[1-(2-oxo-2-phenylethyl)-1,4-dihydropyridin-3-yl]urea (Compound no. 133);

6-methyl-1-[2-oxo-2-(thiophen-2-yl)ethyl]-N′-({1-[2-oxo-2-(thiophen-2-yl)ethyl]-1,4-dihydropyridin-3-yl}carbonyl)-1,4-dihydropyridine-3-carbohydrazide (Compound no. 134);

1-[2-oxo-2-(thiophen-2-yl)ethyl]-N′-(propan-2-ylsulfonyl)-1,4-dihydropyridine-3-carbohydrazide (Compound no. 135);

N,N′-bis{1-[2-oxo-2-(thiophen-2-yl)ethyl]-1,4-dihydropyridin-3-yl}hydrazine-1,2-dicarboxamide (Compound no. 136);

2-[3-(5-benzyl-1,2-oxazol-3-yl)pyridin-1(4H)-yl]-1-(thiophen-2-yl)ethanone (Compound no. 137);

1-[2-(4-benzylpiperidin-1-yl)-2-oxoethyl]-N′-(methylsulfonyl)-1,4-dihydropyridine-3-carbohydrazide (Compound no. 138);

ethyl 1-{[3-{[2-(methylsulfonyl)hydrazinyl]carbonyl}pyridin-1(4H)-yl]acetyl}prolinate (Compound no. 139);

6-hydroxy-N′-(methylsulfonyl)-1-[2-oxo-2-(thiophen-2-yl)ethyl]-1,4,5,6-tetrahydropyridine-3-carbohydrazide (Compound no. 140);

2,6-dihydroxy-N′-(methylsulfonyl)-1-[2-oxo-2-(thiophen-2-yl)ethyl]piperidine-3-carbohydrazide (Compound no. 141);

N′-(methylsulfonyl)-6-oxo-1-[2-oxo-2-(thiophen-2-yl)ethyl]-1,6-dihydropyridine-3-carbohydrazide (Compound no. 142);

1-[2-(4-bromophenyl)-2-oxoethyl]-N′-(methylsulfonyl)-1,4-dihydropyridine-3-carbohydrazide (compound no. 143);

1-[2-(4-methoxyphenyl)-2-oxoethyl]-N′-(methylsulfonyl)-1,4-dihydropyridine-3-carbohydrazide (compound no. 144);

2-[3-({2-[(4-methylphenyl)sulfonyl]hydrazinyl}carbonyl)pyridin-1(4H)-yl]-N-phenylacetamide (compound no. 145);

2-[3-({2-[(4-tert-butylphenyl)sulfonyl]hydrazinyl}carbonyl)pyridin-1(4H)-yl]-N-phenylacetamide (compound no. 146);

N′-[(4-methylphenyl)sulfonyl]-1-[2-oxo-2-(thiophen-2-yl)ethyl]-1,4-dihydropyridine-3-carbohydrazide (compound no. 147);

1-[2-(4-bromophenyl)-2-oxoethyl]-N-(2-hydroxyethyl)-1,4-dihydropyridine-3-carboxamide (compound no. 148);

1-[2-oxo-2-(thiophen-2-yl)ethyl]-N′-(phenylsulfonyl)-1,4-dihydropyridine-3-carbohydrazide (compound no. 149);

N′-[(4-tert-butylphenyl)sulfonyl]-1-[2-oxo-2-(thiophen-2-yl)ethyl]-1,4-dihydropyridine-3-carbohydrazide (compound no. 150);

5-(4-methoxyphenyl)-N′-(methylsulfonyl)-1-[2-oxo-2-(thiophen-2-yl)ethyl]-1,4-dihydropyridine-3-carbohydrazide (compound no. 151);

N′-[(4-methylphenyl)sulfonyl]-1-(2-oxo-2-phenylethyl)-1,4-dihydropyridine-3-carbohydrazide (compound no. 152);

1-[2-oxo-2-(thiophen-2-yl)ethyl]-N′-(thiophen-2-ylcarbonyl)-1,4-dihydropyridine-3-carbohydrazide (compound no. 153);

1-[2-(4-methoxyphenyl)-2-oxoethyl]-N′-[(4-methylphenyl)sulfonyl]-1,4-dihydropyridine-3-carbohydrazide (compound no. 154);

2-{3-[3-(4-bromophenyl)-1,2-oxazol-5-yl]pyridin-1(4H)-yl}-1-(thiophen-2-yl)ethanone (compound no. 155);

2-{3-[3-(4-methylphenyl)-1,2-oxazol-5-yl]pyridin-1(4H)-yl}-N-phenylacetamide (compound no. 156);

N′-[(4-methylphenyl)sulfonyl]-1-[2-oxo-2-(thiophen-2-yl)ethyl]-1,4,5,6-tetrahydropyridine-3-carbohydrazide (compound no. 157);

N-(2-hydroxyethyl)-1-[2-oxo-2-(thiophen-2-yl)ethyl]-1,4,5,6-tetrahydropyridine-3-carboxamide (compound no. 158);

2-[3-({2-[(4-methylphenyl)sulfonyl]hydrazinyl}carbonyl)piperidin-1-yl]-N-phenylacetamide (compound no. 159);

1-[2-oxo-2-(thiophen-2-yl)ethyl]-N′-(phenylsulfonyl)piperidine-3-carbohydrazide (compound no. 160);

N′-[(4-methylphenyl)sulfonyl]-1-(2-oxo-2-phenylethyl)piperidine-3-carbohydrazide (compound no. 161);

1-[2-(4-methoxyphenyl)-2-oxoethyl]-N′-[(4-methylphenyl)sulfonyl]piperidine-3-carbohydrazide (compound no. 162);

1-[2-(4-methoxyphenyl)-2-oxoethyl]-N′-(methylsulfonyl)piperidine-3-carbohydrazide (compound no. 163);

N′-(methylsulfonyl)-1-[2-(5-methylthiophen-2-yl)-2-oxoethyl]piperidine-3-carbohydrazide (compound no. 164);

N′-[(4-tert-butylphenyl)sulfonyl]-1-[2-oxo-2-(thiophen-2-yl)ethyl]piperidine-3-carbohydrazide (compound no. 165);

methyl{3-[5-(4-bromophenyl)-1H-pyrazol-3-yl]piperidin-1-yl}acetate (compound no. 166);

1-[2-(4-bromophenyl)-2-oxoethyl]-N′-(methylsulfonyl)-1,2,5,6-tetrahydropyridine-3-carbohydrazide (compound no. 167);

1-[2-(2,4-dichlorophenyl)-2-oxoethyl]-N′-(methylsulfonyl)-1,4-dihydropyridine-3-carbohydrazide (compound no. 168);

1-[2-oxo-2-(thiophen-2-yl)ethyl]-N′-(phenylsulfonyl)-1,2,5,6-tetrahydropyridine-3-carbohydrazide (compound no. 169);

N′-[(2Z)-2-(ethenylsulfanyl)but-2-enoyl]-1-[2-oxo-2-(thiophen-2-yl)ethyl]-1,2,5,6-tetrahydropyridine-3-carbohydrazide (compound no. 170);

N-(4-chlorophenyl)-2-({1-[2-oxo-2-(thiophen-2-yl)ethyl]-1,2,5,6-tetrahydropyridin-3-yl}carbonyl)hydrazinecarboxamide (compound no. 171);

1-[2-(4-methoxyphenyl)-2-oxoethyl]-N′-(methylsulfonyl)-1,2,5,6-tetrahydropyridine-3-carbohydrazide (compound no. 172);

1-[2-(4-methoxyphenyl)-2-oxoethyl]-N′-[(4-methylphenyl)sulfonyl]-1,2,5,6-tetrahydropyridine-3-carbohydrazide (compound no. 173);

N′-(methylsulfonyl)-1-[2-(5-methylthiophen-2-yl)-2-oxoethyl]-1,2,5,6-tetrahydropyridine-3-carbohydrazide (compound no. 174);

N′-[(4-tert-butylphenyl)sulfonyl]-1-[2-oxo-2-(thiophen-2-yl)ethyl]-1,2,5,6-tetrahydropyridine-3-carbohydrazide (compound no. 175);

2-{5-[3-(4-methoxyphenyl)-1,2-oxazol-5-yl]-3,6-dihydropyridin-1(2H)-yl}-1-(thiophen-2-yl)ethanone (compound no. 176);

methyl{5-[5-(4-bromophenyl)-1H-pyrazol-3-yl]-3,6-dihydropyridin-1(2H)-yl}acetate (compound no. 177);

2-{5-[3-(3-hydroxyphenyl)-1,2-oxazol-5-yl]-3,6-dihydropyridin-1(2H)-yl}-1-(thiophen-2-yl)ethanone (compound no. 178);

2-{5-[5-(4-hydroxyphenyl)-1H-pyrazol-3-yl]-3,6-dihydropyridin-1(2H)-yl}acetamide (compound no. 179);

2-{5-[5-(4-fluorophenyl)-1H-pyrazol-3-yl]-3,6-dihydropyridin-1(2H)-yl}acetamide (compound no. 180);

1-[2-(4-methoxyphenyl)-2-oxoethyl]-N′-(methylsulfonyl)-1,2,3,6-tetrahydropyridine-3-carbohydrazide (compound no. 181);

N′-(methylsulfonyl)-1-[2-(5-methylthiophen-2-yl)-2-oxoethyl]-1,2,3,6-tetrahydropyridine-3-carbohydrazide (compound no. 182);

N′-(methylsulfonyl)-1-[2-oxo-2-(thiophen-2-yl)ethyl]-1,4,5,6-tetrahydropyridine-3-carbohydrazide (compound no. 183);

N′-(methylsulfonyl)-1-[2-oxo-2-(thiophen-2-yl)ethyl]piperidine-3-carbohydrazide (compound no. 184);

N′-(methylsulfonyl)-1-[2-oxo-2-(thiophen-2-yl)ethyl]-1,2,5,6-tetrahydropyridine-3-carbohydrazide (compound no. 185);

N′-(methylsulfonyl)-1-[2-oxo-2-(thiophen-2-yl)ethyl]-1,2,3,6-tetrahydropyridine-3-carbohydrazide (compound no. 186);

N′-(methylsulfonyl)-1-[2-oxo-2-(thiophen-2-yl)ethyl]-1,4,5,6-tetrahydropyridine-3-carbohydrazide (compound no. 187);

2-{3-[3-(3-hydroxyphenyl)-1,2-oxazol-5-yl]pyridin-1(4H)-yl}-1-(thiophen-2-yl)ethanone (compound no. 188);

2-{3-[5-(4-hydroxyphenyl)-1H-pyrazol-3-yl]pyridin-1(4H)-yl}acetamide (compound no. 189);

1-[2-(1-benzofuran-2-yl)-2-oxoethyl]-N′-(methylsulfonyl)-1,2,5,6-tetrahydropyridine-3-carbohydrazide (compound no. 190);

ethyl{5-[3-(4-fluorophenyl)-1H-pyrazol-5-yl]-3,6-dihydropyridin-1(2H)-yl}acetate (compound no. 191);

2-{5-[3-(4-bromophenyl)-1H-pyrazol-5-yl]-3,6-dihydropyridin-1(2H)-yl}acetamide (compound no. 192);

1-[2-(1-benzofuran-2-yl)-2-oxoethyl]-N′-(methylsulfonyl)piperidine-3-carbohydrazide (compound no. 193);

methyl 1-{[3-{[2-(methylsulfonyl)hydrazinyl]carbonyl}pyridin-1(4H)-yl]acetyl}prolinate (compound no. 194);

5-methyl-N′-(methylsulfonyl)-1-[2-oxo-2-(thiophen-2-yl)ethyl]-1,4-dihydropyridine-3-carbohydrazide (compound no. 195);

6-methoxy-N′-(methylsulfonyl)-1-[2-oxo-2-(thiophen-2-yl)ethyl]-1,4-dihydropyridine-3-carbohydrazide (compound no. 196);

N′-(methylsulfonyl)-1-[2-oxo-2-(thiophen-2-yl)ethyl]-6-(pyrrolidin-1-yl)-1,4-dihydropyridine-3-carbohydrazide (compound no. 197);

6-chloro-N′-(methylsulfonyl)-1-[2-oxo-2-(thiophen-2-yl)ethyl]-1,4-dihydropyridine-3-carbohydrazide (compound no. 198);

2-methyl-N′-(methylsulfonyl)-1-[2-oxo-2-(thiophen-2-yl)ethyl]-1,4-dihydropyridine-3-carbohydrazide (compound no. 199);

2-(methylsulfanyl)-N′-(methylsulfonyl)-1-[2-oxo-2-(thiophen-2-yl)ethyl]-1,4-dihydropyridine-3-carbohydrazide (compound no. 200);

2-(dimethylamino)ethyl 1-[2-oxo-2-(thiophen-2-yl)ethyl]-1,4-dihydropyridine-3-carboxylate (compound no. 201);

2-ethoxyethyl 1-[2-oxo-2-(thiophen-2-yl)ethyl]-1,4-dihydropyridine-3-carboxylate (compound no. 202);

4-(5-{1-[2-oxo-2-(phenylamino)ethyl]-1,4-dihydropyridin-3-yl}-1H-pyrazol-3-yl)phenyl (compound no. 203);

4-(5-{1-[2-oxo-2-(phenylamino)ethyl]-1,4-dihydropyridin-3-yl}-1-phenyl-1H-pyrazol-3-yl)phenyl (compound no. 204);

2-[({1-[2-oxo-2-(phenylamino)ethyl]-1,4-dihydropyridin-3-yl}carbonyl)amino]ethyl 4-methoxybenzoate (compound no. 205);

6-methoxy-N′-(methylsulfonyl)-1-[2-oxo-2-(thiophen-2-yl)ethyl]-1,4,5,6-tetrahydro pyridine-3-carbohydrazide (compound no. 206) and its pharmaceutically acceptable salt.

In another embodiment, the present invention provides a new class of heterocyclic compounds of the formula (I) and pharmaceutically acceptable salt thereof,

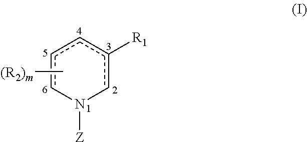

wherein, the dotted line in nitrogen containing ring represents:
(a) two double bond between either (i) at C2-C3 and C5-C6, or (ii) at C2-C3 and C4-C5, or (iii) at C3-C4 and C5-C6, or
(b) one double between either (i) at C2-C3 or (ii) at C3-C4 or (iii) at C4-C5 or (iv) or C5-C6, or
(c) absence of double bond i.e. a saturated ring system;

R₁ is —COR₃ or 5 membered heterocyclic ring having the following formula;

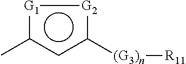

G₁ & G₂ are independently N, NH, NR₁₂, S or O to form heterocyclic ring system, which may also be either partially or fully saturated;

G₃ is —(C₁-C₁₂)alkylene-P or —(C₁-C₁₂)alkylene, wherein P is sulfur, oxygen or nitrogen, and n is 0 or 1;

Z is i) —CH₂—C(O)—Rₓ or ii) R_{y};

Rₓ is R₇, OR₇, —N(R₇)(R₁₀); —N(R₇)N(R₇)(R₁₀), —CH(R₇)C(O)R₈ or a compound having one of the following formula

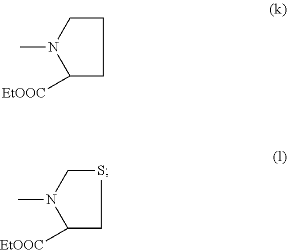

R_{y} is linear or branched (C₁-C₁₂)alkyl;

R₂ is at each occurrence halogen, OR₇, alky, aryl, heterocyclyl, oxo, or —SR₇;

m is 0 or 1;

R₃ is —R₄—R₅, —N(R₇)N(R₇)R₉ or a compound having one of the formula (a), (b), (c), (d), (e), (f), (g), (h), (i) or (j) as defined herein above;

R₄ is —N(R₇)R₆O—, —OR₆O— or —OR₆N(R₇) where R₆ is alkylene;

R₅ is hydrogen, alkyl, —COR₇ or COR₁₀;

R₇ is H, alkyl aryl or heterocyclyl;

R₈ is selected from R₇, OR₇ or NR₇R₁₀;

R₉ is selected from the group consisting of hydrogen, aryl, heterocyclyl, —C(O)R₁₀, —SO₂R₁₀ and C(O)NHR₁₀;

R₁₀ is selected from the group consisting of H, alkyl, alkoxy, aryl and heterocyclyl;

R₁₁ is selected from the group consisting of linear or branched (C₁-C₁₂)alkyl, (C₃-C₇)cycloalkyl, aryl, aralkyl, heterocyclyl, heterocycloalkyl and a compound (m),

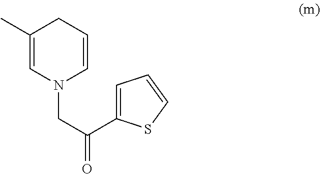

wherein in R₁₁ one or more heteroatoms when present are independently O, N, or S and is optionally substituted, wherein the substituents are selected from a first group consisting of halogen, hydroxy, nitro, cyano, amino, oxo and oxime or from a second group consisting of linear or branched (C₁-C₈)alkyl, (C₃-C₇)cycloalkyl, alkylcycloalkyl, perhaloalkyl, perhalocycloalkyl, aryl, aralkyl, alkylaryl, alkylheterocyclyl, aralkoxylalkyl, perhaloaryl, alkylheterocycloalkyl, heterocycloalkyl, perhaloheterocycloalkyl, heterocyclyl, perhaloheteroaryl, alkoxyalkyl, thioalkyl and thioaryl, wherein the substitutents from said second group are optionally substituted by halogen, hydroxy, nitro, cyano, amino, oxo, perhaloalkyl (C₁-C₆) and oxime and are optionally and independently bridged by —CO, —(CO)O—, —(CO)NH—, —NH—, —NR₁₄—, —O—, —S—, —(SO)—, —(SO₂), —(SO₂)NH—, or —NH(CO)—;

R₁₂ and R₁₃ are independently selected from the group consisting of linear or branched (C₁-C₈)alkyl, (C₃-C₇)cycloalkyl, alkylcycloalkyl, aryl and heterocyclyl, wherein members of said group are optionally substituted by R₁₆;

R₁₄ and R₁₅ are independently selected from the group consisting of linear or branched (C₁-C₁₂)alkyl, (C₃-C₇)cycloalkyl, bicycloalkyl, aryl, and heterocyclyl wherein substituents of said group are optionally substituted by R₁₆;

R₁₆ is halogen, hydroxy, nitro, cyano, amino, oxo, perhaloalkyl (C₁-C₆), or oxime; with the proviso that

(i) when R₁ is —C(O)R₃, then Z is —CH₂—C(O)-Rx.

(ii) when Z is —CH2—C(O)—Rₓ and Rₓ is OR₇, then R₇ is not hydrogen.

Another embodiment of the present invention provides a new class of heterocyclic compounds of the formula (I) and pharmaceutically acceptable salt thereof,

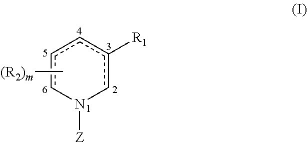

wherein, the dotted line in nitrogen containing ring having double bond at C2-C3 and C5-C6;

R₁ is —COR₃ or 5 membered heterocyclic ring having the following formula;

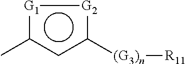

G₁ & G₂ are independently N, NH, NR₁₂, S or O to form heterocyclic ring system, which may also be either partially or fully saturated;

G₃ is —(C₁-C₁₂)alkylene-P or —(C₁-C₁₂)alkylene, wherein P is sulfur, oxygen or nitrogen, and n is 0 or 1;

Z is i) —CH₂—C(O)—Rₓ or ii) R_{y};

Rₓ is R₇, OR₇, —N(R₇)(R₁₀), —N(R₇)N(R₇)(R₁₀), —CH(R₇)C(O)R₈ or a compound having one of the following formula,

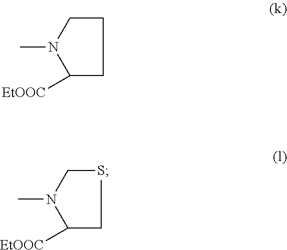

R_{y} is linear or branched (C₁-C₁₂)alkyl;

R₂ is each occurrence from halogen, OR₇, alky, aryl, heterocyclyl, oxo or —SR₇;

m is 0 or 1;

R₃ is —R₄—R₅, —N(R₇)N(R₇)R₉ or a compound having one of the formula (a), (b), (c), (d), (e), (f), (g), (h), (i) or (j) as defined herein above;

R₄ is —N(R₇)R₆O—, —OR₆O— or —OR₆N(R₇) where R₆ is alkylene;

R₅ is hydrogen, alkyl, —COR₇ or COR₁₀;

R₇ is H, alkyl aryl or heterocyclyl;

R₈ is R₇, OR₇ or NR₇R₁₀;

R₉ is selected from the group consisting of hydrogen, aryl, heterocyclyl, —C(O)R₁₀, —SO₂R₁₀ and C(O)NHR₁₀;

R₁₀ is selected from the group consisting of H, alkyl, alkoxy, aryl and heterocyclyl;

R₁₁ is selected from the group consisting of linear or branched (C₁-C₁₂)alkyl, (C₃-C₇)cycloalkyl, aryl, aralkyl, heterocyclyl, heterocycloalkyl and a compound (m),

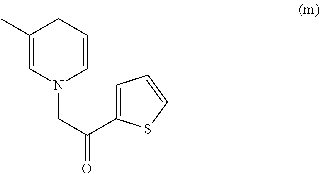

wherein in R₁₁ one or more heteroatoms when present are independently O, N, or S and is optionally substituted, wherein the substituents are selected from a first group consisting of halogen, hydroxy, nitro, cyano, amino, oxo and oxime or from a second group consisting of linear or branched (C₁-C₈)alkyl, (C₃-C₇)cycloalkyl, alkylcycloalkyl, perhaloalkyl, perhalocycloalkyl, aryl, aralkyl, alkylaryl, alkylheterocyclyl, aralkoxylalkyl, perhaloaryl, alkylheterocycloalkyl, heterocycloalkyl, perhaloheterocycloalkyl, heterocyclyl, perhaloheteroaryl, alkoxyalkyl, thioalkyl and thioaryl, wherein the substitutents from said second group are optionally substituted by halogen; hydroxy, nitro, cyano, amino, oxo, perhaloalkyl (C₁-C₆) and oxime and are optionally and independently bridged by —CO, —(CO)O—, —(CO)NH—, —NH—, —NR₁₄—, —O—, —S—, —(SO)—, —(SO₂), —(SO₂)NH—, or —NH(CO)—;

R₁₂ and R₁₃ are independently selected from the group consisting of linear or branched (C₁-C₈)alkyl, (C₃-C₇)cycloalkyl, alkylcycloalkyl, aryl and heterocyclyl, wherein members of said group are optionally substituted by R₁₆;

R₁₄ and R₁₅ are independently selected from the group consisting of linear or branched (C₁-C₁₂)alkyl, (C₃-C₇)cycloalkyl, bicycloalkyl, aryl, and heterocyclyl wherein substituents of said group are optionally substituted by R₁₆;

R₁₆ is halogen, hydroxy, nitro, cyano, amino, oxo, perhaloalkyl (C₁-C₆), or oxime; with the proviso that

(i) when R₁ is —C(O)R₃, then Z is —CH₂—C(O)-Rx.

(ii) when Z is —CH2-C(O)—Rₓ and Rₓ is OR₇, then R₇ is not hydrogen.

In another embodiment, the present invention provides a new class of heterocyclic compounds of the formula (I) and pharmaceutically acceptable salt thereof,

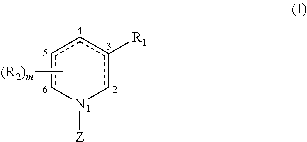

wherein, the dotted line in nitrogen containing ring represents:
(a) two double bonds at C2-C3 and C5-C6, or
(b) one double between either (i) at C2-C3 or (ii) at C3-C4 or (iii) at C4-C5, or
(c) absence of double bond i.e. a saturated ring system;

R₁ is —COR₃;

Z is —CH₂—C(O)—Rₓ;

Rₓ is R₇, OR₇, —N(R₇)(R₁₀); —N(R₇)N(R₇)(R₁₀) or CH(R₇)C(O)R_{8;}

R₂ is aryl and m is 0 or 1;

R₃ is —R₄—R₅ or —N(R₇)N(R₇)R₉;

R₄ is —N(R₇)R₆O—, —OR₆O— or —OR₆N(R₇) where R₆ is alkylene;

R₅ is hydrogen, alkyl, —COR₇ or COR₁₀;

R₇ is H, alkyl aryl or heterocyclyl;

R₈ is R₇, OR₇ or NR₇R₁₀;

R₉ is selected from the group consisting of hydrogen, aryl, heterocyclyl, —C(O)R₁₀, —SO₂R₁₀ and C(O)NHR₁₀;

R₁₀ is selected from the group consisting of H, alkyl, alkoxy, aryl and heterocyclyl; with the proviso that;

when Z is —CH2-C(O)—Rₓ and Rₓ is OR₇, then R₇ is not hydrogen.

In another embodiment, the present invention provides a compound selected from the group comprising of:

1-[2-(5-chlorothiophen-2-yl)-2-oxoethyl]-N′-(methylsulfonyl)-1,4-dihydropyridine-3-carbohydrazide (Compound no. 6);

N′-(methylsulfonyl)-1-[2-(5-methylthiophen-2-yl)-2-oxoethyl]-1,4-dihydropyridine-3-carbohydrazide (Compound no. 13);

N′-(methylsulfonyl)-1-[2-oxo-2-(thiophen-2-yl)ethyl]-1,4-dihydropyridine-3-carbohydrazide (Compound no. 100);

2-[({1-[2-oxo-2-(phenylamino)ethyl]-1,4-dihydropyridin-3-yl}carbonyl)amino]ethyl benzoate (Compound no. 107);

1-[2-(4-bromophenyl)-2-oxoethyl]-N′-(methylsulfonyl)-1,4-dihydropyridine-3-carbohydrazide (compound no. 143);

1-[2-(4-methoxyphenyl)-2-oxoethyl]-N′-(methylsulfonyl)-1,4-dihydropyridine-3-carbohydrazide (compound no. 144);

2-[3-({2-[(4-methylphenyl)sulfonyl]hydrazinyl}carbonyl)pyridin-1(4H)-yl]-N-phenylacetamide (compound no. 145);

N′-[(4-methylphenyl)sulfonyl]-1-[2-oxo-2-(thiophen-2-yl)ethyl]-1,4-dihydropyridine-3-carbohydrazide (compound no. 147);

1-[2-(4-bromophenyl)-2-oxoethyl]-N-(2-hydroxyethyl)-1,4-dihydropyridine-3-carboxamide (compound no. 148);

5-(4-methoxyphenyl)-N′-(methylsulfonyl)-1-[2-oxo-2-(thiophen-2-yl)ethyl]-1,4-dihydropyridine-3-carbohydrazide (compound no. 151);

N-(2-hydroxyethyl)-1-[2-oxo-2-(thiophen-2-yl)ethyl]-1,4,5,6-tetrahydropyridine-3-carboxamide (compound no. 158);

1-[2-oxo-2-(thiophen-2-yl)ethyl]-N′-(phenylsulfonyl)piperidine-3-carbohydrazide (compound no. 160);

1-[2-(4-methoxyphenyl)-2-oxoethyl]-N′-[(4-methylphenyl)sulfonyl]piperidine-3-carbohydrazide (compound no. 162);

1-[2-(4-methoxyphenyl)-2-oxoethyl]-N′-(methylsulfonyl)piperidine-3-carbohydrazide (compound no. 163);

N′-(methylsulfonyl)-1-[2-(5-methylthiophen-2-yl)-2-oxoethyl]piperidine-3-carbohydrazide (compound no. 164);

N′-[(4-tert-butylphenyl)sulfonyl]-1-[2-oxo-2-(thiophen-2-yl)ethyl]piperidine-3-carbohydrazide (compound no. 165);

1-[2-(4-bromophenyl)-2-oxoethyl]-N′-(methylsulfonyl)-1,2,5,6-tetrahydropyridine-3-carbohydrazide (compound no. 167);

1-[2-(2,4-dichlorophenyl)-2-oxoethyl]-N′-(methylsulfonyl)-1,4-dihydropyridine-3-carbohydrazide (compound no. 168);

1-[2-oxo-2-(thiophen-2-yl)ethyl]-N′-(phenylsulfonyl)-1,2,5,6-tetrahydropyridine-3-carbohydrazide (compound no. 169);

N-(4-chlorophenyl)-2-({1-[2-oxo-2-(thiophen-2-yl)ethyl]-1,2,5,6-tetrahydropyridin-3-yl}carbonyl)hydrazinecarboxamide (compound no. 171);

1-[2-(4-methoxyphenyl)-2-oxoethyl]-N′-(methylsulfonyl)-1,2,5,6-tetrahydropyridine-3-carbohydrazide (compound no. 172);

1-[2-(4-methoxyphenyl)-2-oxoethyl]-N′-[(4-methylphenyl)sulfonyl]-1,2,5,6-tetrahydropyridine-3-carbohydrazide (compound no. 173);

N′-(methylsulfonyl)-1-[2-(5-methylthiophen-2-yl)-2-oxoethyl]-1,2,5,6-tetrahydropyridine-3-carbohydrazide (compound no. 174);

1-[2-(4-methoxyphenyl)-2-oxoethyl]-N′-(methylsulfonyl)-1,2,3,6-tetrahydropyridine-3-carbohydrazide (compound no. 181);

N′-(methylsulfonyl)-1-[2-(5-methylthiophen-2-yl)-2-oxoethyl]-1,2,3,6-tetrahydropyridine-3-carbohydrazide (compound no. 182);

N′-(methylsulfonyl)-1-[2-oxo-2-(thiophen-2-yl)ethyl]-1,4,5,6-tetrahydropyridine-3-carbohydrazide (compound no. 183);

N′-(methylsulfonyl)-1-[2-oxo-2-(thiophen-2-yl)ethyl]piperidine-3-carbohydrazide (compound no. 184);

N′-(methylsulfonyl)-1-[2-oxo-2-(thiophen-2-yl)ethyl]-1,2,5,6-tetrahydropyridine-3-carbohydrazide (compound no. 185);

N′-(methylsulfonyl)-1-[2-oxo-2-(thiophen-2-yl)ethyl]-1,2,3,6-tetrahydropyridine-3-carbohydrazide (compound no. 186);

N′-(methylsulfonyl)-1-[2-oxo-2-(thiophen-2-yl)ethyl]-1,4,5,6-tetrahydropyridine-3-carbohydrazide (compound no. 187); and its pharmaceutically acceptable salt.

In another embodiment, the present invention provides a compound N′-(methylsulfonyl)-1-[2-oxo-2-(thiophen-2-yl)ethyl]-1,4-dihydropyridine-3-carbohydrazide and pharmaceutically acceptable salt thereof.

### DEFINITIONS

The use of the terms “a” and “an” and “the” and similar referents in the context of describing the invention (especially in the context of the following claims) are to be construed to cover both the singular and the plural, unless otherwise indicated herein or clearly contradicted by context.

The term “compound” employed herein refers to any compound encompassed by the generic formula disclosed herein. The compounds described herein may exist as stereoisomers, regioisomers, atropisomer such as double-bond isomers (i.e., geometric isomers). Accordingly, the chemical structures depicted herein encompass all possible stereoisomers of the illustrated compounds including the stereoisomerically pure form (e.g., geometrically pure) and stereoisomeric mixtures. The compounds may also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds of the invention include, but are not limited to ²H, ³H, ¹³C, ¹⁴C, ₁₅N, ¹⁸O, ¹⁷O. Compounds may exist in unsolvated forms as well as solvated forms, including hydrated forms. In general, compounds may be hydrated or solvated. Certain compounds may show polymorphism (polymorph) such as crystalline or amorphous forms. In general, all physical forms are equivalent for the uses contemplated herein and are intended to be within the scope of the present invention.

The term “regioisomer” is a term known to those skilled in the art and is defined in text books such as Organic Synthesis, Smith, M., (McGraw Hill) 1994, page 21, which defines a regioisomer as “two or more molecules with the same empirical formula, but with a different attachment of the atoms (different connectivity)”.

The term “atropisomer” as used herein refers to a stereoisomer where the element of chirality is located on a molecular plane or axis.

As used herein, the term “polymorphs” pertains to a compound having the same chemical formula, the same salt type and having the same form of hydrate/solvate but having different crystallographic properties.

As used herein, the term “hydrates” pertains to a compound having a number of water molecules bonded to the molecule.

As used herein, the term “solvates” pertains to a compound having a number of solvent molecules bonded to the molecule.

“Pharmaceutically acceptable salts” means the compound which is modified by making non-toxic acid or base addition salts thereof, and further refers to pharmaceutically acceptable solvates, including hydrates, of such compound and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising one or more of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, caprate, cyclomate, gluconate, dodecyl sulfate, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and combinations comprising one or more of the foregoing salts.

As used herein, “alkyl” refers to an optionally substituted hydrocarbon group joined by single carbon-carbon bonds and having 1 to 8 carbon atoms joined together. The alkyl hydrocarbon group may be linear, branched or cyclic, saturated or unsaturated. The substituents, if present, are F, Cl, Br, I, N, S, O, hydroxy, cycloalkyl, heterocyclyl and aryl. In one embodiment, no more than three substituents are present.

As used herein, the term “alkylene” refers to an optionally substituted straight or branched chain divalent hydrocarbon radical having the specified number of carbon atoms, for example, as used herein, the terms “C₁-C₃ alkylene” and “C₁-C₆ alkylene” refer to an alkylene group, as defined above, which contains at least 1, and at most 3 or 6, carbon atoms respectively. The substituents, if present, are F, Cl, Br, I, N, S, O and aryl.

The term “alkenyl”, used either alone or in attachment with another group refers to an unsaturated (═) aliphatic hydrocarbon radical having the indicated number of carbon atoms and that is unsubstituted or optionally substituted. For example, a “C₃-C₆ alkenyl” would refer to any alkenyl group containing three to six carbons in the structure. Alkenyl may be a straight chain or a branched chain.

The term “alkynyl”, used either alone or in attachment with another group refers to an unsaturated (≡) aliphatic hydrocarbon radical having the indicated number of carbon atoms and that is unsubstituted or optionally substituted. For example, a “C₃-C₆ alkynyl” would refer to any alkenyl group containing three to six carbons in the structure. Alkynyl may be a straight chain or a branched chain.

The “alkoxy” refers to an alkyl group as defined above attached to the parent molecular moiety through an oxygen bridge. Representative alkoxy radicals include methoxy, ethoxy, n-propoxy, n-butoxy, n-pentyloxy, n-hexyloxy, sec-butoxy, tert-butoxy, tert-pentyloxy, and the like.

The “cycloalkyl” refers to a saturated aliphatic hydrocarbon radical having the indicated number of carbon atoms and that is unsubstituted or optionally substituted. For example, a “C₃-C₆ cycloalkyl” would refer to any cycloalkyl group containing three to six carbons in the structure.

As used herein, the term “cycloalkenyl” refers to a non-aromatic monocyclic carbocyclic ring having the specified number of carbon atoms and up to 3 carbon-carbon double bonds. “Cycloalkenyl” includes by way of example cyclopentenyl and cyclohexenyl.

The term “aryl” refers to an aromatic group for example, which is a 6 to 10 membered monocyclic or bicyclic ring system, which may be unsubstituted or substituted. Representative aryl groups may be phenyl, naphthyl and the like. When said ring is substituted, the substituents are selected from the group consisting of halogen (e.g., F, Cl, Br, I), hydroxy, alkyl and alkoxy.

The term “heterocyclyl” as used herein, refers to a mono-, bi- or tricyclic hydrocarbon radical which is unsaturated or fully or partially saturated ring system contains one or more, preferably 1 to 3, heteroatoms selected from O, N or S and preferably contains from 3 to 18 ring atoms, which may be substituted or unsubstituted. The term “heterocyclyl” also includes “heteroaryl” moieties. When said ring system is substituted, the substituents are selected from the group consisting of halogen (e.g., F, Cl, Br, I), alkyl, hydroxyl, amino, ester, nitro and alkoxy.

As used herein, the term “halo” or “halogen” denotes a fluoro, chloro, bromo, or iodo group.

The term “bicycloalkyl” as used herein, refers to an alkyl that has its carbon atoms arranged into two rings. Examples include decahydronaphthyl, norbornyi, and bicyclo[2.2.2]octyl.

The term “bicycloalkenyl” as used herein, refers to an alkenyl that has its carbon atoms arranged into two rings. Examples include norbornenyl and 5,6,7,8-octahydronaphthyl.

The term “perhaloalkyl” means, unless otherwise stated, alkyl substituted with (2m′+1) halogen atoms, where m′ is the total number of carbon atoms in the alkyl group.

As used herein, the term “thioalkyl” refers to the moiety —S-alkyl-, wherein alkyl is as defined above.

The term “thioaryl” as used herein, alone or in combination, refers to a radical of formula aryl-S—, wherein the term “aryl” is as defined above.

All substituents (R₁, R₂ . . . ) and their further substituents described herein may be attached to the main structure at any heteroatom or carbon atom which results in formation of stable compound.

As used herein, the term “mammal” means a human or an animal such as monkeys, primates, dogs, cats, horses, cows, etc.

In the context of the present specification, the term “treat” or “treatment” also includes “prophylaxis” unless there are specific indications to the contrary. The term “treat” or “treatment” within the context of the present invention further encompasses to administer a therapeutically effective amount of a compound of the present invention, to mitigate either a pre-existing disease state, acute or chronic, or a recurring condition. This definition also encompasses prophylactic therapies for prevention of recurring condition and continued therapy for chronic disorders.

The phrase “a therapeutically effective amount” means the amount of a compound that, when administered to a patient for treating a disease, is sufficient to effect such treatment for the disease. The “therapeutically effective amount” will vary depending on the compound, mode of administration, the disease and its severity and the age, weight, etc., of the patient to be treated.

Throughout this specification and the appended claims it is to be understood that the words “comprise” and “include” and variations such as “comprises”, “comprising”, “includes”, “including” are to be interpreted inclusively, unless the context requires otherwise. That is, the use of these words may imply the inclusion of an element or elements not specifically recited.

The nomenclature of the compounds of the present invention as indicated herein is according to ACD/Labs. Name Pro-Version 12.0 from ACD/Lab of Advanced Chemistry Development Inc.

Pharmaceutical Composition:

In another embodiment of the invention is provided a pharmaceutical composition comprising a therapeutically effective amount of one or more of a compound of formula (I) and pharmaceutically acceptable salt thereof and one or more pharmaceutically excipient(s) or other media as may be appropriate for the purpose. While it is possible to administer therapeutically effective quantity of compound of formula (I) either individually or in combination, directly without any formulation, it is common practice to administer the compounds in the form of pharmaceutical dosage forms comprising pharmaceutically acceptable excipient(s) and at least one active ingredient. These dosage forms may be administered by a variety of routes including oral, topical, transdermal, subcutaneous, intramuscular, intravenous, intranasal, pulmonary, buccal, sublingual, etc.

Oral compositions may be in the form of solid or liquid dosage form. Solid dosage form may comprise pellets, pouches, sachets or discrete units such as tablets, multi-particulate units, capsules (soft & hard gelatin) etc. Liquid dosage forms may be in the form of elixirs, suspensions, emulsions, solutions, syrups etc. The above pharmaceutical compositions may contain in addition to active ingredients, excipients such as diluents, disintegrating agents, binders, solubilizers, lubricants, glidants, surfactants, suspending agents, emulsifiers, chelating agents, alkalizing agent, stabilizers, flavours, sweeteners, colours etc. Some example of suitable excipients include lactose, cellulose and its derivatives such as microcrystalline cellulose, methylcelullose, hydroxy propyl methyl cellulose, ethylcellulose, dicalcium phosphate, mannitol, starch, gelatin, polyvinyl pyrrolidone, various gums like acacia, tragacanth, xanthan, alginates & its derivatives, sorbitol, dextrose, xylitol, magnesium stearate, talc, colloidal silicon dioxide, mineral oil, glyceryl mono stearate, glyceryl behenate, sodium starch glycolate, cross povidone, crosslinked carboxymethylcellulose, various emulsifiers such as polyethylene glycol, sorbitol fattyacid, esters, polyethylene glycol alkylethers, sugar esters, polyoxyethylene polyoxypropyl block copolymers, polyethoxylated fatty acid monoesters, diesters and mixtures thereof.

The alkalizing agent may be one or more of amino acids, amino acid esters, diisopropylethylamine, ethanolamine, ethylenediamine, triethanolamine, meglumine, trimethylamine, triethylamine, triisopropanolamine and salts of pharmaceutically acceptable acids. It may be one or more inorganic alkalizers like salts of alkali metals and alkaline earth metals.

The buffering agent described herein include but are not limited to sodium acetate, sodium citrate, sodium bicarbonate, sodium tartrate, sodium fumarate, sodium malate, sodium succinate, magnesium oxide, aluminum oxide, dihydroxy aluminum sodium carbonate, an alkaline earth metal hydroxide such as calcium hydroxide or magnesium hydroxide, with sodium acetate, sodium bicarbonate or sodium citrate being preferred.

Sterile compositions for injection can be formulated according to conventional pharmaceutical practice by dissolving or suspending the active substance in a vehicle such as water for injection, N-Methyl-2-Pyrrolidone, propylene glycol and other glycols, alcohols, a naturally occurring vegetable oil like sesame oil, coconut oil, peanut oil, cotton seed oil or a synthetic fatty vehicle like ethyl oleate or the like. Buffers, anti-oxidants, preservatives, wetting agent, complexing agents like cellulose derivatives, peptides, polypeptides and cyclodextrins and the like can be incorporated as required.

The dosage form can have a slow, delayed or controlled release of active ingredients in addition to immediate release dosage forms.

The amount of active ingredient which is required to achieve a therapeutic effect will, of course, vary with the particular compound, the route of administration, the subject under treatment, and the particular disorder or disease being treated. The compound of the invention may be administered orally or parenterally at a dose of from 0.001 to 1500 mg/kg per day, from 0.01 to 1500 mg/kg per day, from 0.1 to 1500 mg/kg per day, most preferably from 0.1 to 500 mg/kg per day. Tablets or other forms of presentation provided in discrete units may conveniently contain an amount of compound of the invention which is effective at such dosage or as a multiple of the same, for example units containing 1 mg to 1500 mg, usually around 1 mg to 500 mg.

In another embodiment, the pharmaceutical composition of the present invention is an acid resistant formulation like enteric coated formulation of compound of present disclosure or pharmaceutical composition comprising one or more buffering agent and/or one or more alkalizing agent.

In another embodiment, the pharmaceutical composition of the present invention is a fast dissolving formulation of compound of present disclosure comprising one or more solubilizing agents selected from surface active agents (non-ionic, anionic, cationic), complexing agents (cyclodextrin), hydrophilic polymers (cellulosic polymers, povidone, copovidone, NaCMC, etc), pH modifiers. The fast dissolving formulation can be prepared by direct compression, dry granulation, wet granulation, extrusion, melt granulation, solid dispersion, spray drying, fluid bed granulation, hot-melt extrusion, co-precipitation etc.

Whilst a compound of the invention may be used as the sole active ingredient in a medicament, it is also possible for the compound to be used in combination with one or more further active agents. Such further active agents may be further compounds according to the invention, or they may be different therapeutic agents, for example another AGE breaker/inhibitor, anti diabetic agent, anti-obesity agent, anti-hypertensive or anti-dyslipidemic agent or other pharmaceutically active material.

Another embodiment of the present invention relates to the process for preparing compounds of formula (I).

The following reaction scheme-I is given to disclose the synthesis of the compounds according to the present Invention.

Accordingly, the compounds of the present invention may be prepared as described in the scheme-I below.

The compound of general formula (I) includes, but is not limited to, compounds of formula (Ia), (Ib), (Ic), (Id), (Ie), (If), and (Ig) are obtained through the intermediate (II), (III) and (IV), wherein the R₁, R₂ & Z are as defined above and X is halogen.

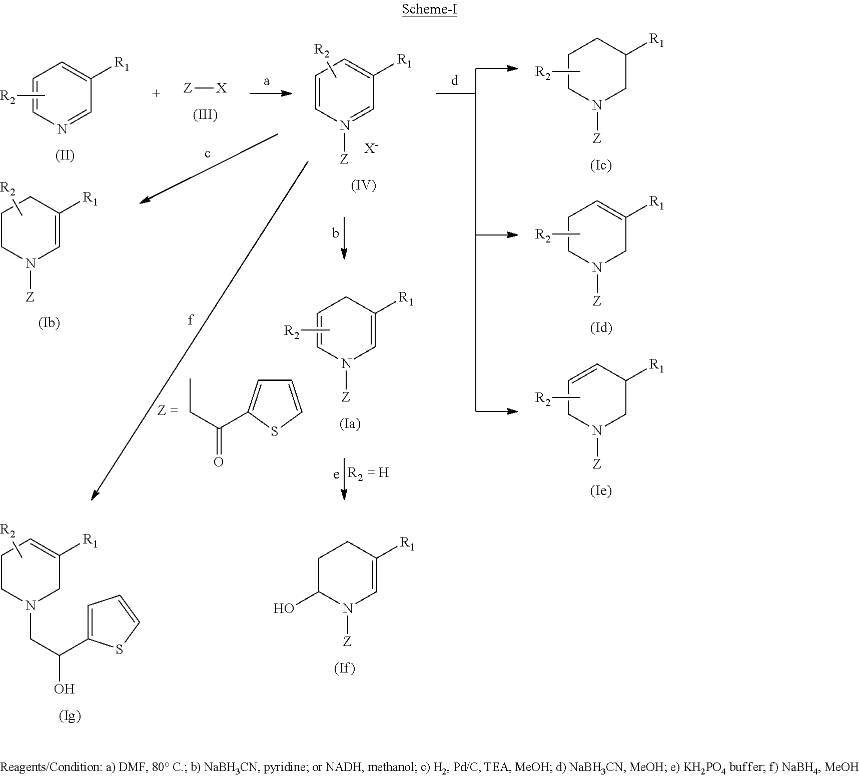

The compound of the formula (II) and (III) is either commercially available or can be prepared by the process known in the prior art.

a) The compound of formula (II) is reacted with suitable halide of formula (III) using similar conditions as described in WO/01/25209 A1 to give the compound of formula (IV).

b) The compound of formula (Ia) is prepared by the reduction of compounds of formula (IV) with suitable reducing agents like sodium cyanoborohydride in a suitable solvent system such as pyridine, tetrahydrofuran, dimethylformamide, 2,6-lutidine, 2-chloropyridine, 4-methoxypyridine, dichloromethane, diglyme, quinoline, dimethylsulfoxide, sulfolane, 2-methoxyethanol, dimethylacetamide, or combination of 1,2-dimethoxy ethane (DME) with one or more solvent selected from the group comprising of dioxane, pyridine, nitromethane, water or DMF.

The compound of formula (Ia) is also prepared by the reduction of compound of formula (IV) with nicotinamide adenine dinucleotide hydrogen (NADH) in suitable solvent such as methanol.

In another way, the compound of formula (Ia) can also be prepared by the reduction of compound of formula (IV) with sodium dithionite in the presence of base such sodium carbonate, potassium carbonate, sodium bicarbonate in suitable solvent such as dichloromethane.

“Further, the process to prepare the 1,4-dihydropyridine of formula (Ia) of the present invention resides in the fact that the reduction can be carried out using above mentioned reducing reagents or other known reducing agents such as lithiumtetrahydroborate, tetrabutylammonium cyanoborohydride, selectride with varying reaction condition such as time, temperature and solvent. In certain conditions, reduction for longer time may yield various substituted tetrahydropyridine or piperidine.”

Further, the 1,4-dihydropyridine compound of formula (Ia) is also purified by technique known in the art such as crystallization from suitable solvent such as acetonitrile, nitromethane, dioxane:isopropanol:1,2-dimethoxyethane, etc.

c) The compound of the formula (Ib) is prepared by catalytic hydrogenation of compound of formula (IV) in a suitable solvents like methanol.

d) The compound of formula (Ic), (Id) and (Ie) is prepared by the reduction of compound of formula (IV) with reducing agents like sodium cyanoborohydride in suitable solvents like methanol.

e) The compound of formula (If) is prepared by the reaction of compound of formula (Ia) with phosphate buffer, which could be further converted to corresponding methyl derivatives.

f) The compound of formula (Ig) is prepared by the reduction of compound of formula (IV) with suitable reducing agent such as sodium borohydride in suitable solvent such as methanol.

One of ordinary skill will know to substitute an appropriately modified starting material containing the various substitutents to prepare the desired compound of the present invention using the general synthesis scheme depicted above.

The compounds of the present invention may have chiral centers and occur as racemates, racemic mixture and as individual diastereomers or enantiomers with all isomeric forms being included in the present invention. Therefore, where a compound is chiral, the separate enantiomers, substantially free of the other, are included within the scope of the invention; further included are all mixture of the two enantiomers.

The novel compounds of the present invention are not, however, to be construed as forming the only genus that is considered as the invention, and any combination of the compounds or their moieties may itself form a genus.

The novel compounds of the present invention were prepared according to the procedure of the Scheme-I as described herein above, using appropriate materials and are further exemplified by the following specific examples. The Examples are not be considered nor construed as limiting the scope of the invention.

### EXAMPLES

### Example 1

### Preparation of N′-(methylsulfonyl)-1-[2-oxo-2-(thiophen-2-yl)ethyl]-1,4-dihydro-pyridine-3-carbohydrazide (Compound No. 100)

Method-1

To a stirred suspension of Pyridinium, 3-[[2-(methylsulfonyl)hydrazine]carbonyl]-1-[2-oxo-2-(2-thienyl)ethyl], chloride (200 gm, 0.53 mole) in pyridine (1000 ml), sodium cyanoborohydride (40 gm, 0.64 mole) was added at −10° C. to 0° C. in portion wise manner under nitrogen atmosphere. The obtained reaction mixture was stirred at 25-30° C. for 2 hrs. The separated solid was filtered and suck dried. The solid was dissolved in dichloromethane and washed with water. The organic layer was separated and dried over sodium sulphate and the dichloromethane was evaporated. The separated solid was filtered, washed with dichloromethane and dried under vacuum at 50° C.-55° C. to get desired product (20 gm) as a yellow solid.

¹H NMR (400 MHz, DMSO-d₆)

δ 9.36 (s, 1H), 9.12 (s, 1H), 8.08-8.07 (d, 1H), 8.00-7.99 (d, 1H), 7.30-7.28 (t, 1H), 7.00 (s, 1H), 5.88-5.86 (d, 1H), 4.77 (s, 2H), 4.68-4.64 (m, 1H), 3.03 (s, 2H), 2.89 (s, 3H)

¹³C NMR (DMSO-d₆) δ 21.68, 39.74, 58.46, 97.84, 102.19, 129.14, 130.40, 133.79, 135.53, 140.39, 141.09, 167.34, 189.29

ESMS (m/z): 342 (M+1)

Method -2

### Step-1: Preparation of 3-carboxy-1-[2-oxo-2-(thiophen-2-yl)ethyl]pyridinium chloride

To a stirred solution of Nicotinic acid (5.0 gms, 0.040 mole) in dimethyl formamide (50 ml), 2-α-chloroacetylthiophene (8.0 gm, 0.05 mole) was added and the reaction mixture was stirred for 14 hrs at 80° C. for 15 hrs. The reaction mixture was cooled to 25-28° C. The diethyl ether was added to mixture and separated solid was filtered. The crude product was purified with mixture of methanol and ethyl acetate to provide 4.2 gram of the desired product as a solid.

### Step-2: Preparation of 1-[2-oxo-2-(thiophene-2-yl)ethyl]-1,4-dihydropyridine-3-carboxylic acid

To a stirred suspension of 3-carboxy-1-[2-oxo-2-(thiophen-2-yl)ethyl]pyridinium chloride (4.2 gm, 0.017 mole) in pyridine (50 ml), sodium cyanoborohydride (2.0 gm, 0.032 mole) was added at −10° C. to 0° C. portion wise under nitrogen atmosphere. Reaction mixture was stirred at 25-30° C. for 2 hrs. The separated solid was filtered and suck dried. The solid was dissolved in dichloromethane and washed with water. The organic layer was separated and dried over sodium sulphate and the dichloromethane was evaporated. The separated solid was filtered and washed with dichloromethane and dried under vacuum at 50-55° C. to get the desire product (0.8 gm) as a yellowish colored solid.

### Step-3: Preparation of N′-(methylsulfonyl)-1-[2-oxo-2-(thiophen-2-yl)ethyl]-1,4-dihydro-pyridine-3-carbohydrazide (Compound No. 100)

To a solution of 1-[2-oxo-2-(thiophen-2-yl)ethyl]-1,4-dihydropyridine-3-carboxylic acid (0.5 gm, 0.002 mole) in dichloromethane (20 ml) at 0° C. was added 1-(3-dimethylaminopropyl)-3-ethylcarbodiimide hydrochloride (EDCI) (0.462 gm, 0.0024 mole) followed by the addition of 1-hydroxybenzotriazole (0.27 gm, 0.002 mole) and triethylamine (0.85 ml, 0.006 mole) and the reaction mixture was stirred for 30 min. To the above solution, methane sulfonohydrazide (0.231 gm, 0.0021 mole) was added and the reaction mixture was stirred for 8 hr. Then after water (20 ml) was added to the reaction mixture and washed with saturated sodium bicarbonate solution (2×20 ml) and finally with water (20 ml). The methylene chloride layer was dried over sodium sulphate and evaporated under vacuum to yield crude product, which was purified over silica gel using Ethylacetate: Hexanes as a eluent to give 0.1 gm of the desire product as a solid.

Upon the characterization of obtained solid product, spectrographic analysis showed that a desired compound was obtained as a mixture with other impurities.

### Example 2

### Alternative Process for the Preparation of N′-(methylsulfonyl)-1-[2-oxo-2-(thiophen-2-yl)ethyl]-1,4-dihydro-pyridine-3-carbohydrazide (Compound No. 100)

To a stirred cold solution of 1,2-dimethoxyethane (600 ml) and Pyridinium, 3-[[2-(methylsulfonyl)hydrazino]carbonyl]-1-[2-oxo-2-(2-thienyl)ethyl]chloride (120 gm, 0.32 mole), sodium cyanoborohydride (30 gm, 0.48 mole) was added at −10° C. to 0° C. and stirred at ambient temperature for 2-3 hrs. The obtained crude was filtered and washed with water. The crude was stirred in water:ethanol (1:1) for 1 hour and filtered. The crude product was dried under vacuum for 15 hrs. at 55° C. to get desired product (61 gm).

The crude product (3 gm) was further purified by recrystallization in acetonitrile to give the title product (1.6 gm).

### Example 3

### Preparation of N′-(methylsulfonyl)-1-[2-oxo-2-(thiophen-2-yl)ethyl]-1,4,5,6-tetrahydropyridine-3-carbohydrazide (Compound No. 183)

To a mixture of 10% Pd/C (1 g, 20% w/w) in methanol (25 ml), triethyl amine (0.47 ml, 3.18 mmol) was added at room temperature under nitrogen atmosphere. Then pyridinium, 3-[[2-(methylsulfonyl)hydrazino]carbonyl]-1-[2-oxo-2-(2-thienyl)ethyl]chloride (1 g, 2.65 mmol) was added to the above mixture in portions. A hydrogen atmosphere (50 mbar) was applied and reaction continued at room temperature for 50 hrs. The reaction mixture filtered through hyflow, the filtrate was distilled, and the residue was suspended in water. The solid, thus obtained, was filtered and dried under vacuum. The crude product thus obtained, was purified further by silica gel column chromatography using ethyl acetate and hexane as eluent to yield a yellow solid product (0.18g).

¹H NMR (400 MHz, DMSO-d₆) δ 1.78 (2H, s), 2.18 (2H,s), 2.88 (3H, s), 3.11 (2H, s), 4.73 (2H, s), 7.30 (2H, s), 8.00-8.07 (2H, m), 8.93 (1H, s), 9.17 (1H, s)

¹³C NMR (DMSO-d₆) δ 19.62, 21.05, 39.96, 46.70, 60.76, 95.35, 129.13, 133.74, 135.45, 141.33, 144.77, 167.75, 189.88

ESMS (m/z) 344 (M+1)

### Example 4

To the suspension of Pyridinium, 3-[[2-(methylsulfonyl)hydrazino]carbonyl]-1-[2-oxo-2-(2-thienyl)ethyl]chloride (2 gm., 5.3 mmol) in methanol (25 ml), sodium cyanoborohydride (0.492 g, 7.95 mmol) was added in portions at room temperature. The reaction mixture was stirred at room temperature for 6 h. The reaction mixture was poured into water and extracted by ethyl acetate. The crude product, thus obtained, was purified further by silica gel column chromatography in an ethyl acetate and hexane mixture. Following three compounds were isolated.

### N′-(methylsulfonyl)-1-[2-oxo-2-(thiophen-2-yl)ethyl]piperidine-3-carbohydrazide (Compound No. 184)

¹H NMR (400 MHz, DMSO-d₆) δ 1.39-1.51 (2H, m), 1.63-1.74 (2H, m), 2.18 (1H, m), 2.32 (1H, m), 2.50 (1H, m, partially overlapped with solvent peak), 2.76-2.83 (2H, m), 2.88 (3H, s), 3.72 (2H, s), 7.24 (1H, s), 7.99-8.03 (2H, m), 9.38 (1H, s), 10.19 (1H, s)

ESMS (m/z) 344 (M−1), 346 (M+1)

### N′-(methylsulfonyl)-1-[2-oxo-2-(thiophen-2-yl)ethyl]-1,2,5,6-tetrahydropyridine-3-carbohydrazide (Compound No. 185)

¹H NMR (400 MHz, DMSO-d₆) δ 2.27 (2H, m), 2.64 (2H, t), 2.91 (3H, s), 3.22 (2H, bs), 3.87 (2H, s), 6.70 (1H, bs), 7.23 (1H, m), 7.98-8.03 (2H, m), 9.39 (1H, s), 10.18 (1H, s)

¹³C NMR (DMSO-d₆) δ 25.76, 40.60, 48.67, 50.96, 64.06, 128.66, 130.33, 132.64, 133.75, 135.17, 142.04, 166.02, 190.81

ESMS (m/z) 342 (M−1), 344 (M+1)

### N′-(methylsulfonyl)-1-[2-oxo-2-(thiophen-2-yl)ethyl]-1,2,3,6-tetrahydropyridi-ne-3-carbohydrazide (Compound No. 186)

¹H NMR (400 MHz, DMSO-d₆) δ 2.76-2.81 (1H, m), 2.85-2.86 (m, 1H), 2.89 (3H, s), 3.01-3.11 (2H, m), 3.15-3.20 (1H, m), 3.88 (2H, d), 5.70 (1H, dd), 5.83 (1H, dd), 7.24 (1H, t), 8.03 (2H, dd), 9.42 (1H, d), 10.22 (1H, d)

ESMS (m/z) 342 (M−1), 344 (M+1)

### Example 5

### Preparation of 6-hydroxy-N′-(methylsulfonyl)-1-[2-oxo-2-(thiophen-2-yl)ethyl]-1,4,5,6-tetrahydropyridine-3-carbohydrazide (Compound No. 140)

N′-(methylsulfonyl)-1-[2-oxo-2-(2-thienyl)ethyl]-1,4-dihydropyridine-3-carbohydrazide (2 g, 5.86 mmol) was stirred in freshly prepared KH₂PO₄ buffer (5.44 gm in 200 ml water) at room temperature for 8 days. The reaction mixture was basified by sodium bicarbonate and extracted by ethyl acetate. The ethyl acetate layer was dried over sodium sulfate and concentrated under vacuum. The crude product was purified using the Waters auto purification system (Preparative HPLC system) to yield 0.25 g of title product as a white solid.

¹H NMR (400 MHz, DMSO-d₆) δ 1.59-1.62 (1H, m), 1.75-1.82 (1H, m), 2.07-2.18 (1H, m), 2.25-2.32 (1H, m), 2.89 (3H, s), 4.65-4.92 (3H, m), 5.77 (1H, d), 7.20 (1H, s), 7.29 (1H, t), 8.02 (1H, d), 8.06 (1H, d), 8.99 (1H, bs), 9.34 (1H, s)

¹³C NMR (DMSO-d₆) δ 15.21, 28.03, 39.75, 57.73, 77.12, 97.05, 129.09, 133.59, 135.36,141.35, 142.74, 167.44, 190.01

ESMS (m/z) −360 (M+1)

### Example 6

### Preparation of N′-(methylsulfonyl)-1-[2-oxo-2-(thiophen-2-yl)ethyl]-1,4,5,6-tetrahydropyridine-3-carbohydrazide (Compound No. 187)

To the suspension of Pyridinium, 3-[[2-(methylsulfonyl)hydrazino]carbonyl]-1-[2-oxo-2-(2-thienyl)ethyl]chloride (2 g, 5.3 mmol) in methanol (25 ml), sodium borohydride (0.251 g, 6.62 mmol) was added in portions at room temperature. The reaction mixture was stirred at room temperature for 6 h. The reaction mixture was poured into water and extracted by ethyl acetate. The crude product, thus obtained, was purified further by silica gel column chromatography in ethyl acetate and hexane mixture.

¹H NMR (400 MHz, DMSO-d₆) δ 2.23 (2H, m), 2.64 (4H, m), 2.92 (3H, s), 3.20 (2H, s), 5.00 (1H, q), 5.51 (1H, d), 6.67 (1H, s), 6.94-6.98 (2H, m), 7.36 (1H, d), 9.39 (1H, s), 10.14 (1H, s)

¹³C NMR (DMSO-d₆) δ 25.64, 40.64, 49.30, 51.85, 65.92, 66.64, 123.40, 124.50, 126.67, 130.35, 132.95, 149.07, 166.02

ESMS (m/z) −346 (M+) 344 (M−1)

The following representative compounds of the present invention were prepared in analogues manner by following the synthetic route as described above:

TABLE 1

| Comp. | | |
|---|---|---|
| No. | ¹H-NMR (400 MHz, DMSO-d₆) | ESMS (m/z) |
| 13 | δ 2.53 (3H, s, partially overlapped with solvent | 356 (M + 1) |
| | peak), 2.89 (3H, s), 3.02 (2H, s), 4.64-4.69 (3H, | |
| | m), 5.85 (1H, d), 7.01 (2H, m), 7.81 (1H, d), | |
| | 9.13 (1H, s), 9.36 (1H, s), | |
| 6 | δ 2.89 (3H, s), 3.01 (2H, s), 4.64-4.68 (1H, m), | 374 (M − 1) |
| | 4.74 (2H, s), 5.85 (1H, d), 6.98 (1H, s), 7.35 (1H, | |
| | d), 7.90 (1H, d), 9.13 (1H, d), 9.36 (1H, d) | |
| 143 | δ 2.89 (3H, s), 3.04 (2H, s), 4.66 (1H, m), 4.79 | 412 (M − 1) |
| | (2H, s), 5.84 (1H, d), 6.98 (1H, s), 7.78 (2H, d), | 414 (M − 1) |
| | 7.88 (2H, d), 9.12 (1H, s), 9.37 (1H, s) | |
| 144 | δ 2.89 (3H, s), 3.03 (2H, s), 3.89 (3H, s), 4.63- | 366 (M + 1) |
| | 4.66 (1H, m), 4.79 (2H, s), 5.83 (1H, d), 6.88 | |
| | (1H, s), 7.07 (2H, d), 7.93 (2H, d), 9.12 (1H, s), | |
| | 9.33 (1H, s) | |
| 145 | δ 2.33 (3H, s), 2.89 (2H, s), 3.95 (2H, s), 4.60 | 427 (M + 1) |
| | (1H, m), 5.82 (1H, d), 6.82 (1H, s), 7.05 (1H, t), | 425 (M − 1) |
| | 7.33-7.29 (4H, m), 7.57 (2H, d), 7.67 (2H, d), | |
| | 9.28 (1H, s), 9.38 (1H, s), 10.03 (1H, s) | |
| 107 | δ 3.01 (2H, s), 3.48 (2H, q), 3.98 (2H, s), 4.28 | 404 (M − 1) |
| | (2H, t), 4.59-4.63 (1H, m), 5.87 (1H, d), 6.90 (1H, | 406 (M + 1) |
| | s), 7.05 (1H, t), 7.29-7.33 (3H, m), 7.50-7.66 | |
| | (5H, m), 7.98 (2H, m), 10.06 (1H, s) | |
| 146 | δ 1.28 (9H, s), 2.89 (2H, s), 3.93 (2H, s), 4.61 | 469 (M + 1) |
| | (1H, m), 5.84 (1H, d), 6.85 (1H, s), 7.05 (1H, t), | |
| | 7.31 (2H, t), 7.50-7.59 (5H, m), 7.72 (2H, m), | |
| | 9.27 (1H, s), 10.04 (1H, s) | |
| 147 | δ 2.37 (3H, s), 2.91 (2H, s), 4.59-4.62 (1H, m), | 416 (M − 1) |
| | 4.72 (2H, s), 5.81 (1H, dd), 6.81 (1H, d), 7.28- | |
| | 7.33 (3H, m), 7.65 (2H, d), 7.97 (1H, dd), 8.07 | |
| | (1H, dd), 9.30 (1H, s), 9.38 (1H, s) | |
| 148 | δ 3.02 (2H, s), 3.17 (2H, d), 4.01-4.05 (1H, d), | 366 (M + 1) |
| | 4.62-4.73 (2H, m), 4.82 (2H, s), 5.82 (1H, d), | |
| | 6.84 (1H, s), 6.94 (1H, m), 7.78 (2H, d), 7.88 | |
| | (2H, d) | |
| 149 | δ 2.91 (2H, s), 4.58-4.62 (1H, m), 4.72 (2H, s), | 402 (M − 1) |
| | 5.81 (1H, dd), 6.79 (1H, s), 7.29 (1H, m), 7.51 | |
| | (2H, m), 7.61 (1H, m), 7.79 (2H, m), 7.96 | |
| | (1H, dd), 8.07 (1H, dd), 9.29 (1H, s), 9.49 (1H, s). | |
| 150 | δ 1.28 (9H, s), 2.91 (2H, s), 4.61 (1H, bs), 4.72 | 460 (M + 1) |
| | (2H, s), 5.82 (1H, d), 6.81 (1H, s), 7.28 (1H, s), | |
| | 7.55 (2H, d), 7.71 (2H, d), 7.90-7.97 (2H, d), 9.24 | |
| | (1H, s), 9.37 (1H, s). | |
| 1 | δ 2.93 (3H, s), 3.38 (2H, m), 4.85 (2H, s), 6.48 | 420 (M + 1) |
| | (1H, s), 7.08 (1H, s), 7.29 (1H, t), 7.99 (1H, d), | |
| | 8.08 (1H, d), 9.11 (1H, s), 9.52 (1H, s). | |
| 151 | δ 2.94 (3H, s), 3.31 (2H, s), 3.75 (3H, s), 4.97 | 448 (M + 1) |
| | (2H, s), 6.70 (1H, s), 6.91 (2H, d), 7.16 (1H, s), | |
| | 7.31-7.37 (3H, m), 8.04-8.12 (2H, dd), 9.24 (1H, | |
| | s), 9.64 (1H, s). | |
| 152 | δ 2.36 (3H, s), 2.94 (2H, s), 4.76 (1H, m), 4.81 | 412 (M + 1) |
| | (2H, s), 5.79 (1H, d), 6.79 (1H, s), 7.32 (2H, d), | |
| | 7.54-7.67 (5H, m), 7.93 (2H, d), 9.25 (1H, s), | |
| | 9.38 (1H, s). | |
| 153 | δ 3.08 (2H, s), 4.67 (1H, m), 4.78 (2H, s), 5.89 | 374 (M + 1) |
| | (1H, d), 6.97 (1H, s), 7.18 (1H, m), 7.29 (1H, t), | 372 (M − 1) |
| | 7.81-7.84 (2H, m), 8.00 (1H, d), 8.08 (1H, d), | |
| | 9.10 (1H, s), 10.21 (1H, s). | |
| 154 | δ 2.36 (3H, s), 2.92 (2H, s), 3.84 (3H, s), 4.57- | 440 (M − 1) |
| | 4.61 (1H, m), 4.74 (2H, s), 5.77 (1H, d), 6.76 | 442 (M + 1) |
| | (1H, s), 7.06 (2H, d), 7.32 (2H, d), 7.67 (2H, d), | |
| | 7.92 (2H, d), 9.23 (1H, s), 9.36 (1H, s). | |
| 155 | δ 3.16 (2H, s), 4.65-4.79 (1H, m), 4.85 (2H, d), | 427 (M⁺) |
| | 5.97-6.01 (1H, m), 6.95 (1H, d), 7.15 (1H, s), | |
| | 7.29 (1H, q), 7.69-7.80 (4H, m), 8.01-8.10 (2H, | |
| | m). | |
| 156 | δ 2.35 (3H, s), 3.15 (2H, s), 4.04-4.09 (2H, m), | 370 (M − 1) |
| | 4.67-4.73 (1H, m), 5.97-6.00 (1H, m), 6.92-6.95 | |
| | (1H, m), 7.03-7.07 (2H, m), 7.27-7.38 (4H, m), | |
| | 7.59-7.61 (2H, d), 7.65 (1H, d), 7.73 (1H, d), | |
| | 10.04 (1H, m). | |
| 157 | δ 1.72 (2H, t), 2.07 (2H, t), 2.34 (3H, s), 3.05 | 418 (M − 1) |
| | (2H, t), 4.70 (2H, s), 7.15-7.41 (3H, m), 7.64- | |
| | 7.78 (3H, m), 7.96 (1H, d), 8.05 (1H, d), 9.04 | |
| | (1H, s),9.12(1H, s). | |
| 158 | δ 1.78 (2H, t), 2.14 (2H, t), 3.04-3.09 (4H, q), | 295 (M + 1) |
| | 3.13-3.17 (2H, q), 4.66 (2H, s), 4.73 (1H, t), 6.86 | |
| | (1H, t), 7.14 (1H, s), 7.28 (1H, t), 8.0 (1H, dd). | |
| 159 | δ 1.54 (2H, m), 2.17-2.44 (6H, m), 2.57 (2H, t), | 431 (M + 1) |
| | 3.05 (2H, m), 3.21 (2H, s), 7.07 (1H, m), 7.24- | |
| | 7.34 (4H, m), 7.62-7.67 (4H, m), 9.72-9.75 (2H, | |
| | m), 10.14 (1H, s). | |
| 160 | δ 1.07-1.16 (1H, m), 1.37-1.46 (1H, m), 1.56 | 408 (M + 1) |
| | (2H, m), 1.98-2.08 (2H, m), 2.31-2.36 (1H, m), | 406 (M − 1) |
| | 2.50-2.73 (2H, dd), 3.65 (2H, s), 7.22-7.25 (1H, | |
| | m), 7.50 (2H, t), 7.58-7.63 (1H, m), 7.76 (2H, d), | |
| | 7.99-8.01 (2H, m), 9.77 (1H, s), 10.13 (1H, s). | |
| 161 | δ 2.09 (1H, m), 2.28 (3H, s), 2.36 (2H, s), 2.58 | 415 (M⁺) |
| | (2H, t), 2.68 (1H, m), 3.02 (2H, s), 3.79 (1H, d), | |
| | 3.94 (2H, m), 7.31 (2H, t), 7.49-7.54 (2H, m), | |
| | 7.63-7.54 (3H, m), 7.96 (2H, t), 9.66 (1H, s), | |
| | 10.17 (1H, s). | |
| 162 | δ 1.19-1.22 (1H, m), 1.38-1.41 (1H, m), 1.52 | 444 (M − 1) |
| | (2H, m), 2.09 (2H, m), 2.29 (3H, s), 2.56 (2H, | 446 (M + 1) |
| | m), 2.68 (1H, m), 3.72 (2H, m), 3.84 (3H, s), | |
| | 7.04 (2H, d), 7.30 (2H, d), 7.64 (2H, d), 7.95 | |
| | (2H, d), 9.65 (1H, s), 10.12 (1H, s). | |
| 163 | δ 1.40-1.47 (2H, m), 1.62-1.71 (2H, m), 2.16 | 368 (M − 1) |
| | (1H, t), 2.31 (1H, t), 2.43 (1H, m), 2.73-2.81 | 370 (M + 1) |
| | (2H, dd), 2.88 (3H, s), 3.77 (2H, d), 3.84 (3H, s), | |
| | 7.03 (2H, d), 7.98 (2H, d), 9.38 (1H, s), 10.20 | |
| | (1H, s). | |
| 164 | δ 1.38-1.56 (2H, m), 1.63-1.66 (1H, m), 1.71- | 360 (M + 1) |
| | 1.73 (1H, m), 2.12-2.17 (1H, m), 2.29 (1H, t), | |
| | 2.51 (4H, m, overlapped with solvent peak), | |
| | 2.73-2.83 (2H, m), 2.88 (3H, s), 3.67 (2H, s), | |
| | 6.96 (1H, d), 7.85 (1H, d), 9.38 (1H, s), 10.18 | |
| | (1H, s). | |
| 165 | δ 1.09-1.25 (1H, m), 1.28 (9H, s), 1.38-1.46 (1H, | 464 (M + 1) |
| | m), 1.51-1.58 (2H, m), 1.95-2.10 (2H, dt), 2.31- | |
| | 2.36 (1H, t), 2.58 (1H, d), 2.71 (1H, d), 3.59-3.71 | |
| | (2H, m), 7.24 (1H, t), 7.52 (2H, d), 7.68 (2H, d), | |
| | 8.00 (2H, m), 9.61 (1H, s), 10.11 (1H, s). | |
| 166 | CDCl₃ δ 1.75-2.31 (4H, m), 3.10-3.48 (6H, m), | 379 (M + 1) |
| | 3.76 (3H, s), 4.28-4.59 (1H, m), 6.32 (1H, s), | |
| | 7.49 (2H, d), 7.62 (2H, d), 11.93 (1H, s). | |
| 167 | δ 2.23 (2H, m), 2.61 (2H, t), 2.89 (3H, s), 3.21 | 415.9 (M⁺) |
| | (2H, s), 3.96 (2H, s), 6.67 (1H, s), 7.7 (2H, d), | 417.9 (M + 1) |
| | 7.9 (2H, d), 9.38 (1H, s), 10.17 (1H, s) | |
| 168 | δ 2.17 (2H, m), 2.58 (2H, t), 2.88 (3H, s), 3.21 | 406 (M⁺) |
| | (2H, s), 3.85 (2H, s), 6.58 (1H, s), 7.5 (1H, dd), | |
| | 7.80 (2H, m), 9.32 (1H, s), 10.17 (1H, s). | |
| 169 | δ 2.21 (2H, m), 2.58 (2H, t), 3.01 (2H, d), 3.81 | 404 (M − 1) |
| | (2H, s), 6.55 (1H, s), 7.23 (1H, t), 7.51 (2H, t), | 406 (M + 1) |
| | 7.61 (1H, t), 7.76 (2H, d), 8.00 (2H, m), 9.78 | |
| | (1H, s), 10.13 (1H, s). | |
| 170 | δ 2.30 (2H, m), 2.67 (2H, t), 3.25 (2H, s), 3.88 | 376 (M + 1) |
| | (2H, s), 6.72 (1H, s), 7.17-7.24 (2H, m), 7.83 | |
| | (2H, d), 8.06 (2H, dd), 9.06 (1H, s). | |
| 171 | δ 2.22 (2H, s), 2.68 (2H, s), 3.19 (2H, s), 3.90 | 417 (M − 1), |
| | (2H, s), 6.72 (1H, s), 7.24 (1H, t), 7.29 (2H, d), | 419 (M + 1) |
| | 7.48 (2H, d), 8.00 (1H, d), 8.03-8.06 (2H, m), | |
| | 8.94 (1H, s), 9.73 (1H, s). | |
| 172 | δ 2.23 (2H, s), 2.61 (2H, t), 2.91 (3H, s), 3.20 | 366 (M − 1) |
| | (2H, s), 3.83 (3H, s), 3.90 (2H, s), 6.67 (1H, s), | 368 (M + 1) |
| | 7.03 (2H, d), 7.98 (2H, d), 9.37 (1H, s), 10.16 | |
| | (1H, s). | |
| 173 | δ 2.18 (2H, s), 2.36 (3H, s), 2.56 (2H, t), 2.99 | 442 (M − 1) |
| | (2H, s), 3.83 (3H, s), 3.85 (2H, s), 6.51 (1H, s), | 444 (M + 1) |
| | 7.03 (2H, d), 7.31 (2H, d), 7.65 (2H, d), 7.96 | |
| | (2H, d), 9.67 (1H, s), 10.07 (1H, s). | |
| 174 | δ 2.25 (2H, m), 2.50 (3H, s, merged with solvent | 356 (M − 1) |
| | peak), 2.63 (2H, t), 2.91 (3H, s), 3.20 (2H, s), | 358 (M + 1) |
| | 3.79 (2H, s), 6.69 (1H, s), 6.95 (1H, d), 7.85 (1H, | |
| | d), 9.38 (1H, s), 10.17 (1H, s). | |
| 175 | δ 1.27 (9H, s), 2.22 (2H, s), 2.60 (2H, t), 3.02 | 462 (M + 1) |
| | (2H, s), 3.81 (2H, s), 6.55 (1H, s), 7.14 (1H, s), | |
| | 7.54 (2H, d), 7.70 (2H, d), 8.00 (2H, m), 9.61 | |
| | (1H, bs), 10.08 (1H, s). | |
| 176 | δ 2.60 (1H, m), 2.87 (1H, m), 3.30 (1H, partially | 381 (M − 1) |
| | merged with water signal), 3.81-3.89 (5H, m), | |
| | 4.19 (2H, m), 4.50 (1H, m), 6.60 (1H, bs), 7.08 | |
| | (2H, m), 7.24-7.28 (2H, m), 7.79 (2H, m), 8.04- | |
| | 8.09 (2H, m). | |
| 177 | CDCl₃ δ 2.34 (2H, m), 2.72 (2H, t), 3.38 (2H, s), | 376 (M − 2), |
| | 3.45 (2H, d), 3.69 (3H, s), 6.19 (1H, s), 6.42 (1H, | 378 (M⁺) |
| | s), 7.44-7.53 (4H, dd), 11.93 (1H, s). | |
| 178 | δ 2.38 (2H, s), 2.76 (2H, t), 3.46 (2H, s), 3.98 | 365 (M⁺), |
| | (2H, s), 6.68 (1H, s), 6.79 (1H, d), 6.96 (1H, s), | |
| | 7.22-7.37 (4H, m), 7.97 (1H, d), 8.05 (1H, d). | |
| 179 | DMSO-d₆ + D₂O δ 2.31 (2H, s), 2.62 (2H, t), | 299 (M + 1) |
| | 3.08 (2H, s), 3.36 (2H, s), 6.32 (1H, s), 6.65 (1H, | |
| | bs), 6.83 (2H, d), 7.58 (2H, s). | |
| 180 | DMSO-d₆ + D₂O δ 2.32 (2H, s), 2.62 (2H, t), | 301 (M + 1) |
| | 3.09 (2H, s), 3.36 (2H, s), 6.35 (1H, s), 6.75 (1H, | |
| | bs), 7.25 (2H, t), 7.81 (2H, t), | |
| 181 | δ 2.80 (2H, m), 2.89 (3H, s), 3.05-3.14 (3H, m), | 366 (M − 1) |
| | 3.84 (3H, s), 3.94 (2H, s), 5.69 (1H, d), 5.83 (1H, | |
| | d), 7.03 (2H, d), 7.98 (2H, d), 9.42 (1H, s), 10.27 | |
| | (1H, s). | |
| 182 | DMSO-d₆ + D₂O δ 2.51 (3H, s), 2.76-2.80 (2H, | 358 (M + 1) |
| | m), 2.91 (3H, s), 3.07 (2H, m), 3.13 (1H, m), | |
| | 3.83 (2H, d), 5.70 (1H, dd), 5.84 (1H, dd), 6.96 | |
| | (1H, d), 7.85(1H, d). | |

### EXAMPLES OF PHARMACEUTICAL COMPOSITIONS

### Example 7

### Solution Formulation with HP β Cyclodextrin

Composition:

TABLE 2

| Sr. No. | Ingredients | % W/V |
|---|---|---|
| 1. | Compound no. 100 | 2.0 |
| 2. | HP β Cyclodextrin | 20.0 |
| 3. | 1N NaOH | — |
| 4. | 1N HCl | — |
| 5. | Water | q.s. |

Process:
a) Compound was dissolved in water by addition of 1N NaOH and HP β cyclodextrin was dissolved in water separately.
b) HP β cyclodextrin solution was added to Compound solution and pH was adjusted to 7.5 with 1N HCl. Volume made up with water.

### Example 8

### Non-Acid Resistant Liquid Formulation

Composition:

TABLE 3

| Sr. No. | Ingredients | % W/V |
|---|---|---|
| 1. | Compound no. 100 | 1.0 |
| 2. | PEG-400 | 50.0 |
| 3. | Purified water | q.s. |

Process:

Compound no. 100 was dissolved in PEG400 and volume made up with purified water.

### Example 9

### Acid Resistant Liquid Formulation

Composition:

TABLE 4

| Sr. No. | Ingredients | % W/V |
|---|---|---|
| 1. | Compound no. 100 | 1.0 |
| | (Micronized) | |
| 2. | Cremophor RH40 | 2.0 |
| 3. | Sodium Bicarbonate | 10.0 |
| 4. | 0.5% Sodium CMC in Water | q.s. |

Process:

Compound was suspended in solution of Cremophor RH40 and sodium bicarbonate in 0.5% W/V sodium CMC solution. Volume made up with 0.5% W/V sodium CMC solution.

PK Profile of Non-Acid Resistant and Acid Resistant Formulations

Single dose pharmacokinetics of non-acid resistant and acid resistant formulations of compound no. 100 was studied in male Wistar rat (280-300 gm n=4 for non-acid resistant & n=5 for acid resistant formulations) after oral administration at a dose of 10 mg/kg. Blood samples were obtained serially from jugular vein cannulated animals at selected time points and concentration of compound no. 100 in plasma was determined by LC-MS/MS. Simultaneously concentration of reference compound-T was also determined. Non-compartmental pharmacokinetic analysis was performed using WinNonlin 5.2. Results are mentioned below:

TABLE 5

| | | | | Reference compound | | |
|---|---|---|---|---|---|---|
| | | | | after administration of | | |
| | Compound no. 100 | | | Compound no. 100 | | |
| | Cmax | Tmax | AUC₀₋ₜ | Cmax | Tmax | AUC₀₋ₜ |
| Composition | (SD) | (SD) | (SD) | (SD) | (SD) | (SD) |
| Non-Acid | 80.72 | 0.25 | 61.0 | 89.79 | 0.44 | 45.0 |
| Resistant | (51.3) | (0) | (27.99) | (73.97) | (0.32) | (40.39) |
| Acid | 1354.2 | 0.25 | 1392.6 | 915.2 | 0.50 | 1456.7 |
| Resistant | (535.3) | (0.0) | (586.5) | (338.3) | (0.3) | (613.5) |

Maximum plasma concentration of compound no. 100 & reference compound-T and corresponding AUC₀₋ₜ after administration of acid resistant formulation was higher in comparison with non-acid resistant formulation.

### Example 10

### Fast Dissolving Enteric Coated Tablets Composition:

TABLE 6

| | | |
|---|---|---|
| Sr No | Ingredients | % W/W |

Core

| | | |
|---|---|---|
| 1 | Compound no. 100 | 39.06 |
| | (Micronized) | |
| 2 | Meglumine | 3.91 |
| 3 | Cellulose, Microcrystalline | 21.68 |
| | (silicified) | |
| 4 | Polyethylene Glycol 6000 | 9.77 |
| 5 | Cremophor RH 40 | 0.78 |
| 6 | Croscarmellose Sodium | 1.95 |
| 7 | Magnesium Stearate | 0.98 |

Sub coat

| | | |
|---|---|---|
| 8 | Hydroxy propyl cellulose | 1.45 |
| 9 | Hydroxy propyl methyl | 1.45 |
| | cellulose | |
| 10 | Talc | 0.51 |
| 11 | Titanium dioxide | 1.29 |
| 12 | Methanol | qs |
| | Methylene chloride | qs |

Enteric coat

| | | |
|---|---|---|
| 13 | Eudragit L 30D -55 | 10.74 |
| 14 | Triethyl citrate | 1.09 |
| 15 | Talc | 5.35 |
| 16 | Purified water | Qs |

Compound, MCC and meglumine were mixed together and granulated with hot melt PEG6000 containing Cremophor RH 40. The granules obtained were then mixed with Croscarmellose Sodium and lubricated with Magnesium Stearate. The blend was then compressed into tablets. Tablets were then coated with subcoating and enteric coating composition.

Biological Activity:

In vitro Prevention of Advanced Glycation Endproducts (AGEs) Accumulation:

Proteins when incubated in the presence of reducing sugar undergo nonenzymatic glycosylation (termed as Maillard reaction) to form advanced glycosylation end products (AGEs) that exhibit a characteristic fluorescence spectrum, which can be used to detect their formation. A reduction in fluorescence intensity in the presence of the test compound is an indication of its ability to prevent accumulation of advanced glycation endproducts.

Bovine Serum Albumin was incubated with ribose alone as well as with ribose and different concentrations of the following compounds under aseptic conditions for one week. At the end of the incubation period fluorescence intensity of the samples was measured at excitation and emission wavelengths of 355 nm and 460 nm respectively. Results for the test compound were expressed as % AGEs accumulation, considering the extent of AGEs formation upon incubation with ribose alone as 100%.

TABLE 7

| | | |
|---|---|---|
| | | % Inhibition |
| Compound | | of AGE-BSA |
| No. | Conc. | Formation |

| | | | |
|---|---|---|---|
| 100 | 1 | mM | ++ |
| 13 | 1 | mM | +++ |
| 6 | 1 | mM | +++ |
| 185 | 1 | mM | + |
| 187 | 1 | mM | + |
| 143 | 100 | uM | + |
| 167 | 1 | mM | + |
| 144 | 1 | mM | + |
| 145 | 250 | uM | + |
| 107 | 100 | uM | + |
| 147 | 100 | uM | +++ |
| 183 | 1 | mM | +++ |
| 148 | 1 | mM | + |
| 151 | 100 | uM | + |
| 168 | 100 | uM | + |
| 169 | 100 | uM | + |
| 160 | 100 | uM | + |
| 158 | 250 | uM | + |
| 171 | 100 | uM | + |
| 181 | 250 | uM | + |
| 163 | 250 | uM | + |
| 172 | 50 | uM | + |
| 162 | 100 | uM | + |
| 173 | 100 | uM | + |
| 182 | 1 | mM | + |
| 164 | 1 | mM | + |
| 174 | 1 | mM | + |
| 165 | 100 | uM | + |
| 186 | 1 | mM | + |
| 184 | 1 | mM | + |

+ indicate 0-10%; ++ indicate 11-20%; +++ indicate 21-30%; ++++ indicate >30%

In vitro Inhibition of AGE-LDL Formation:

Low density lipoproteins (LDL) undergo metal catalysed oxidation upon incubation with CuCl₂ to form malondialdehyde (MDA)—lysine adducts that exhibit a characteristic fluorescence spectrum. A reduction in the fluorescence intensity in the presence of the test compound is an indication of their ability to prevent lipid peroxidation.

Low density lipoprotein (LDL) was incubated with CuCl₂ at 37° C. in phosphate buffered saline in the presence and absence of the following compounds. The extent of MDA—lysine adduct formation was quantitated by measuring the fluorescence intensity at excitation and emission wavelengths of 355 nm and 460 nm respectively. Results were expressed as % MDA-lysine adducts accumulation, considering the extent of the same formed upon incubation with CuCl₂ alone as 100%.

TABLE 8

| | | % Inhibition |
|---|---|---|
| Compound | | of AGE-LDL- |
| No. | Conc. | formation |
| 100 | 250 Um | ++++ |
| 13 | 250 uM | ++++ |
| 6 | 250 uM | ++++ |
| 185 | 250 uM | +++ |
| 187 | 250 uM | ++ |
| 143 | 100 uM | ++++ |
| 167 | 250 uM | + |
| 144 | 250 uM | ++++ |
| 145 | 250 uM | + |
| 107 | 100 uM | + |
| 147 | 100 uM | ++++ |
| 183 | 250 uM | ++ |
| 148 | 250 uM | + |
| 151 | 100 uM | + |
| 168 | 100 uM | + |
| 169 | 100 uM | + |
| 160 | 100 uM | + |
| 158 | 100 uM | + |
| 171 | 100 uM | + |
| 181 | 100 uM | + |
| 163 | 100 uM | + |
| 172 | 50 uM | + |
| 162 | 100 uM | ++++ |
| 173 | 100 uM | + |
| 182 | 250 uM | +++ |
| 164 | 250 uM | + |
| 174 | 250 uM | ++++ |
| 165 | 100 uM | + |
| 186 | 250 uM | ++++ |
| 184 | 250 uM | +++ |

+ indicate 0-10%; ++ indicate 11-20%; +++ indicate 21-30%; ++++ indicate >30%

Summary

Compounds of the present invention have shown the ability to prevent the accumulation of AGEs as well as to prevent AGE-LDL accumulation as seen in Table 7 & 8.

Summary of Oral Pharmacokinetics of Reference Compound-T and Compound No. 100 in Wistar Rat:

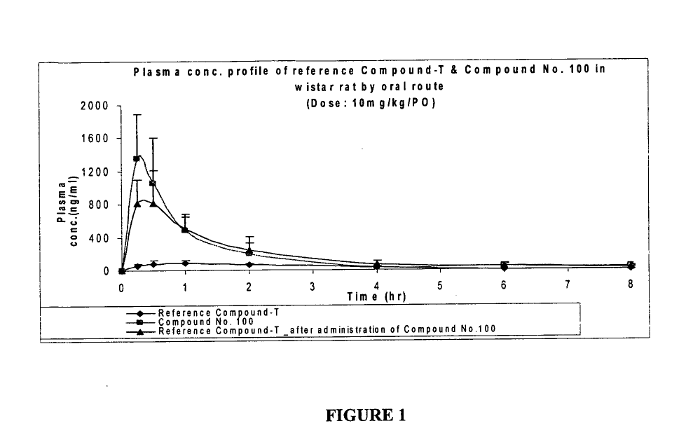

Single dose pharmacokinetics of Reference compound-T (3-((2-(methylsulfonyl)hydrazino)carbonyl)-1-(2-oxo-2-thienylethyl)pyridinium chloride) and compound no. 100 was studied in male Wistar rat (280-300 gm, n=5) after oral administration at a dose of 10 mg/kg. Blood samples were obtained serially from jugular vein cannulated animals at selected time points and concentration of Reference compound-T and compound no. 100 in plasma was determined by LC-MS/MS. Non-compartmental pharmacokinetic analysis was performed using WinNonlin 5.2. Both the compounds were absorbed rapidly. Reference compound was also simultaneously monitored in plasma samples after oral administration of compound no. 100. The pharmacokinetic data is presented below in a tabular and graphical form (Table-9 & FIG. 1):

TABLE 9

| | | | | | | Reference | |
|---|---|---|---|---|---|---|---|
| | | | | | | compound- | |
| | | | | | | T _after | |
| | | Ref. | | | | administration | |
| | | Compound- | | Compound | | of Compound | |
| PK | | T | | No. 100 | | no. 100 | |
| Parameters | Unit | Mean | SD | Mean | SD | Mean | SD |
| Cmax | ng/mL | 100.5 | 30.9 | 1354.2 | 535.3 | 915.2 | 338.3 |
| AUC_{(0-8 hr)} | hr * ng/mL | 203.6 | 31.7 | 1410.6 | 592.9 | 1413.4 | 616.8 |

Observation:
Maximum plasma concentration (Cmax) of reference.compound converted after administration of compound no. 100 was 9 times and corresponding AUC_{(0-8 hr)} was 6.9 times in comparison to the administration of ref. compound.

Summary of Oral Pharmacokinetics of Reference Compound-T and Compound No. 100 in Dog:

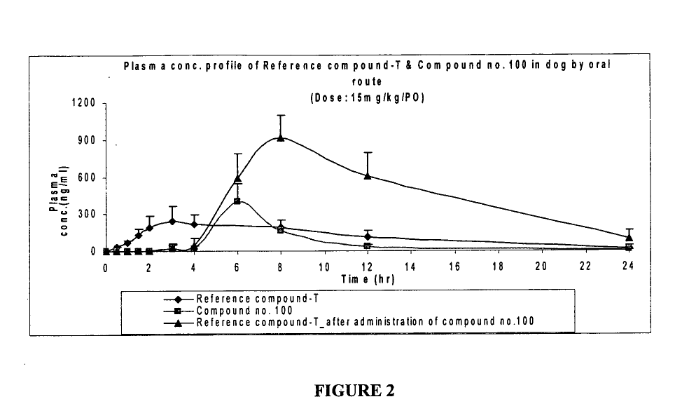

Single dose pharmacokinetics of Reference compound-T (3-((2-(methylsulfonyl)hydrazino)carbonyl)-1-(2-oxo-2-thienylethyl)pyridinium chloride) and compound no. 100 was studied in male dog (n=2 for reference compound & n=3 for compound no. 100) after oral administration at a dose of 15 mg/kg. Blood samples were obtained serially from cephalic vein at selected time points and concentration of reference compound-T and compound no. 100 in plasma was determined by LC-MS/MS. Non-compartmental pharmacokinetic analysis was performed using WinNonlin 5.2. Reference compound was also simultaneously monitored in plasma samples after oral administration of compound no. 100. The pharmacokinetic data is presented below in a tabular and graphical form (Table-10 & FIG. 2):

TABLE 10

| | | | | | | Reference | |
|---|---|---|---|---|---|---|---|
| | | | | | | compound- | |
| | | | | | | T _after | |
| | | Ref. | | | | adminstration | |
| | | Compound- | | Compound | | of Compound | |
| PK | | T | | No. 100 | | no. 100 | |
| Parameters | Unit | Mean | SD | Mean | SD | Mean | SD |
| Cmax | ng/ml | 258.4 | 97.6 | 397.1 | 143.4 | 948.9 | 125.7 |
| AUC_{(0-24 hr)} | hr * ng/ml | 2477.0 | 409.4 | 1780.7 | 850.6 | 8784.6 | 1607.4 |

Observation:
Maximum plasma concentration of reference compound converted after administration of compound no. 100 is approx. 3.6 times and corresponding AUC_{(0-24 hr)} is 3.5 times in comparison to the administration of ref. compound.

### Tissue Distribution of Compound of Present Invention

Reference compound-T (3-((2-(methylsulfonyl)hydrazino)carbonyl)-1-(2-oxo-2-thienylethyl)pyridinium chloride) and Compound No. 100 were administered to male and female wistar rats (n=3) at dose level 100 mg/kg/twice a day for 14 days by intra peritoneal route. On day 15, after morning dose selected tissues were harvested at 0.5 hrs and 6 hours for analysis of reference compound-T. The tissues considered for analysis were liver, kidney, heart, brain and aorta. The tissue homogenate samples were analyzed using LC-MS/MS method. The data is presented in following table-11:

TABLE 11
Ratio of tissue levels of Reference
compound-T after adminstration of
Compound No. 100 to Reference compound-T

| | Time | Tissues | | | | |
|---|---|---|---|---|---|---|
| | (hours) | Liver | Kidney | Heart | Brain | Aorta |
| Male | 0.5 | 0.5 | 0.4 | 6.8 | 5.6 | 2.1 |
| | 6 | 1.8 | 8.9 | 3.0 | 11.9 | 1.9 |
| Female | 0.5 | 2.2 | 0.3 | 4.5 | 10.9 | 1.4 |
| | 6 | 3.0 | 11.4 | 3.8 | NE | 0.8 |

NE = Not evaluable

Observation: The tissue distribution data at 0.5 hour revealed that heart, brain and aorta had higher ratio (>1) i.e. more Reference compound available after administration of Compound No. 100. At Subsequent time points i.e. at 6 hours, this ratio was higher in most of the organs including liver and kidney. This observation indicates the probability of enhanced distribution of compound no. 100 in tissue compartment and subsequent conversion into Reference compound. Availability at later time points suggests longer exposure at tissue/organ level to Reference compound as well.

The enhanced bioavailability of Reference compound in both, the intravascular and tissue compartments address the constraint currently encountered for Reference compound namely comparatively poor bioavailability in both, the vascular and thereafter in the tissue compartment which is also an important site of action

1. N′-(methylsulfonyl)-1-[2-oxo-2-(thiophen-2-yl)ethyl]-1,4-dihydropyridine-3-carbohydrazide and pharmaceutically acceptable salt thereof.

2. A compound of formula (I) and pharmaceutically acceptable salt thereof; 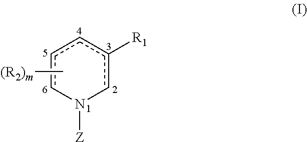
wherein, the dotted line in nitrogen containing ring represents:
(a) two double bond between either (i) at C₂-C₃ and C₅-C₆, or (ii) at C₂-C₃ and C₄-C₅, or (iii) at C₃-C₄ and C₅-C₆, or
(b) one double between either (i) at C₂-C₃ or (ii) at C₃-C₄ or (iii) at C₄-C₅ or (iv) or at C₅-C₆, or
(c) absence of any double bonds;
R₁ is —COR₃ or 5 membered heterocyclic ring having the following formula; 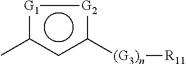
G₁ & G₂ are independently N, NH, NR₁₂, S or O to form heterocyclic ring system, which may also be either partially or fully saturated;
G₃ is —(C₁-C₁₂)alkylene-P or —(C₁-C₁₂)alkylene, wherein P is sulfur, oxygen or nitrogen, and n is 0 or 1;
Z is i) —CH₂—C(O)—R_{X} or ii) R_{Y};
R_{X} is R₇, OR₇, —N(R₇)(R₁₀), —N═C(R₇)(R₁₀), —N(R₇)N(R₇)(R₁₀), —N(R₇)N═C(R₇)(R₁₀), —CH(R₇)C(O)R₈ or a compound having one of the following formula: 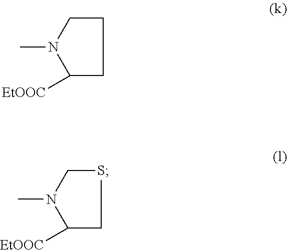
R_{Y} is selected from the group consisting of hydrogen, linear or branched (C₁-C₁₂)alkyl, (C₂-C₁₂)alkenyl, (C₃-C₇)cycloalkyl, (C₅-C₇)cycloalkenyl, bicycloalkyl, CH₂(CO)R₁₃, CH₂(CO)NHR₁₄, CH₂(CO)NR₁₄R₁₅ and CH₂(CO)OR₁₃;
R₂ at each occurrence is halogen, OR₇, NO₂, alkyl, aryl, heterocyclyl, formyl, oxo, —NR₇R₁₀, —N═C(R₇)(R₁₀), —SR₇, —SO₂NH₂, —SO₂ alkyl, —SO₂ aryl, N═C(R₁₄)(R₁₅), —NR₁₄R₁₅, —OR₁₄, perhaloalkyl, —O(CO)R₁₄, —NH(CO)R₁₄, (C₂-C₁₂)alkenyl, (C₃-C₇)cycloalkyl, (C₅-C₇)cycloalkenyl, bicycloalkyl, bicycloalkenyl, heterocycloalkyl, or aralkyl; m is 0, 1, 2 or 3;
R₃ is —R₄—R₅, —N(R₇)N(R₇)R₉ or a compound having one of the following formula: 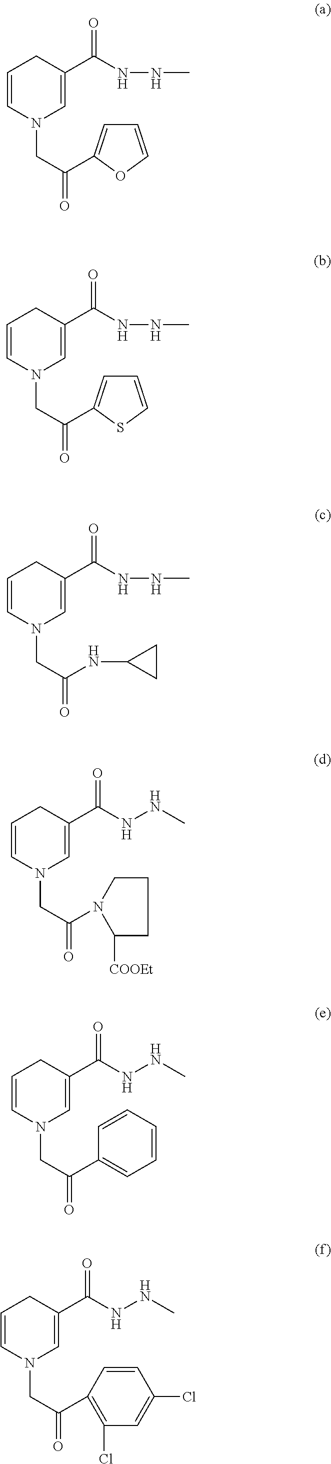 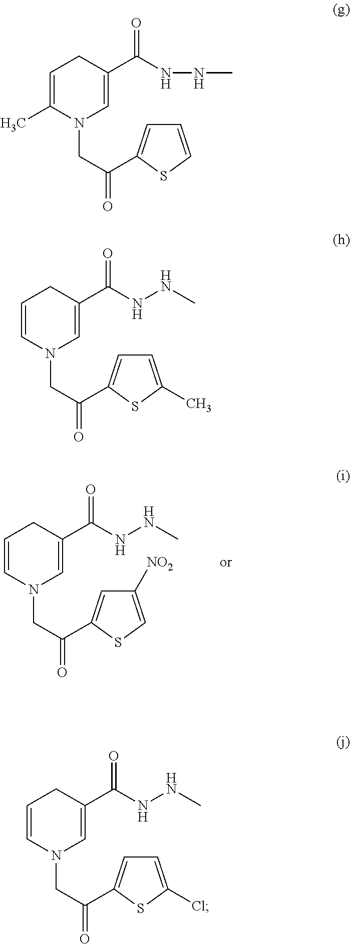
R₄ is —N(R₇)R₆O—, —N(R₇)R₆N(R₇)—, OR₆O or —OR₆N(R₇)—, where R₆ is alkylene;
R₅ is hydrogen, alkyl, aryl, heterocyclyl-COR₇, SO₂R₇, —C(S)NHR₇, —C(═NH)NHR₇, —COR₁₀, —C(O)NHR₇ or 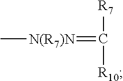
where R₇ is H, alkyl, aryl or heterocyclyl;
R₈ is R₇, OR₇ Or NR₇R₁₀;
R₉ is selected from the group consisting of hydrogen, alkyl, aryl, heterocyclyl, C(O)R₁₀, —SO₂R₁₀, —C(S)NHR₁₀, —C(═NH)NH(R₁₀) and —C(O)NHR₁₀;
R₁₀ is selected from the group consisting of H, alkyl, alkoxy, aryl and heterocyclyl;
R₁₁ is selected from the group consisting of hydrogen, linear or branched (C₁-C₁₂)alkyl, (C₂-C₁₂)alkenyl, (C₃-C₇)cycloalkyl, (C₅-C₇)cycloalkenyl, bicycloalkyl, bicycloalkenyl, heterocycloalkyl, aryl, aralkyl, heterocyclyl and compound (m), 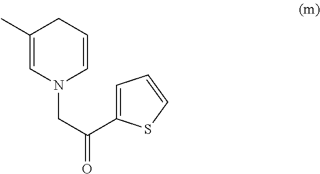
wherein in R₁₁ one or more heteroatoms when present are independently O, N, or S and is optionally substituted, wherein the substituents are selected from a first group consisting of halogen, hydroxy, nitro, cyano, amino, oxo and oxime or from a second group consisting of linear or branched (C₁-C₈)alkyl, (C₃-C₇)cycloalkyl, alkylcycloalkyl, perhaloalkyl, perhalocycloalkyl, aryl, aralkyl, alkylaryl, aralkoxylalkyl, perhaloaryl, alkylheterocycloalkyl, heterocyclyloalkyl, perhaloheterocyclyloalkyl, heterocyclyl, perhaloheteroaryl, alkoxyalkyl, thioalkyl and thioaryl, wherein the substituents from said second group are optionally substituted by halogen, hydroxy, nitro, cyano, amino, oxo, perhaloalkyl (C₁-C₆) and oxime and are optionally and independently bridged by —CO, —(CO)O—, —(CO)NH—, —NH—, —NR₁₄—, —O, —S—, —(SO)—, —(SO₂), —(SO₂)NH—, or —NH(CO)—;
R₁₂ and R₁₃ are independently selected from the group consisting of linear or branched (C₁-C₈)alkyl, (C₃-C₇)cycloalkyl, alkylcycloalkyl, perhaloalkyl, perhalocycloalkyl, aryl, aralkyl, alkylaryl, aralkoxylalkyl, perhaloaryl, alkylheterocycloalkyl, heterocycloalkyl, perhaloheterocycloalkyl, heterocyclyl, perhaloheterocyclyl, —COalkyl, —COaryl, benzoyl, alkoxyalkyl, thioalkyl and thioaryl wherein members of said group are optionally substituted by R₁₆;
R₁₄ and R₁₅ are independently selected from the group consisting of linear or branched (C₁-C₁₂)alkyl, alkoxyaryl, alkoxyalkyl, alkoxycycloalkyl, alkoxyaryl, perhaloalkyl, (C₂-C₁₂)alkenyl, (C₃-C₇)cycloalkyl, perhalocycloalkyl, haloheterocycloalkyl, cyanoheterocycloalkyl, perhaloheterocycloalkyl, (C₅-C₇)cycloalkenyl, bicycloalkyl, bicycloalkenyl, heterocycloalkyl, aryl, aralkyl, heterocyclyl, perhaloaryl and perhaloheterocyclyl wherein substituents of said group are optionally substituted by R₁₆;
R₁₆ is halogen, hydroxy, nitro, cyano, amino, oxo, perhaloalkyl (C₁-C₆), or oxime;
with the proviso that
(i) when R₁ is —C(O)R₃, then Z is —CH₂—C(O)—R_{X}; and
(ii) when Z is —CH2-C(O)—R_{X} and R_{X} is OR₇ then R₇ is not hydrogen.

3. The compound of formula (I) as claimed in claim 2, 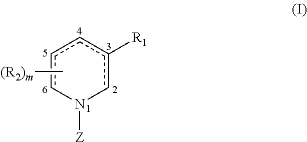
wherein, the dotted line in nitrogen containing ring represents:
(a) two double bond between either (i) at C₂-C₃ and C₅-C₆, or (ii) at C₂-C₃ and C₄-C₅, or (iii) at C₃-C₄ and C₅-C₆, or
(b) one double between either (i) at C₂-C₃ or (ii) at C₃-C₄ or (iii) at C₄-C₅ or (iv) or at C₅-C₆, or
(c) absence of any double bonds;
R₁ is —COR₃ or 5 membered heterocyclic ring having the following formula; 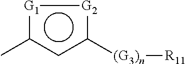
G₁ & G₂ are independently N, NH, NR₁₂, S or O to form heterocyclic ring system, which may also be either partially or fully saturated;
G₃ is —(C₁-C₁₂)alkylene-P or —(C₁-C₁₂)alkylene, wherein P is sulfur, oxygen or nitrogen, and n is 0 or 1;
Z is i) —CH₂—C(O)—R_{X} or ii) R_{Y};
R_{X} is R₇, OR₇, —N(R₇)(R₁₀), —N(R₇)N(R₇)(R₁₀), —CH(R₇)C(O)R₈ or a compound having one of the following formula: 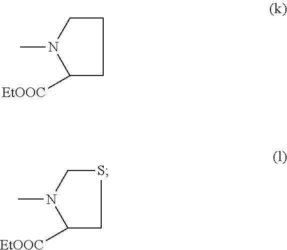
R_{Y} is linear or branched (C₁-C₁₂)alkyl;
R₂ is at each occurrence halogen, OR₇, alky, aryl, heterocyclyl, oxo or —SR₇; m is 0 or 1;
R₃ is —R₄—R₅, —N(R₇)N(R₇)R₉ or a compound having one of the formula (a), (b), (c), (d), (e), (f), (g), (h), (i) or (j);
R₄ is —N(R₇)R₆O—, —OR₆O— or —OR₆N(R₇), where R₆ is alkylene;
R₅ is hydrogen, alkyl, —COR₇ or COR₁₀;
R₇ is H, alkyl aryl or heterocyclyl;
R₈ is R₇, OR₇ or NR₇R₁₀;
R₉ is selected from the group consisting of hydrogen, aryl, heterocyclyl, —C(O)R₁₀, —SO₂R₁₀ and C(O)NHR₁₀;
R₁₀ is selected from the group consisting of H, alkyl, alkoxy, aryl and heterocyclyl;
R₁₁ is selected from the group consisting of linear or branched (C₁-C₁₂)alkyl, (C₃-C₇)cycloalkyl, aryl, aralkyl, heterocyclyl, heterocycloalkyl and a compound (m), 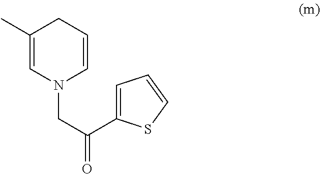
wherein in R₁₁ one or more heteroatoms when present are independently O, N, or S and is optionally substituted, wherein the substituents are selected from a first group consisting of halogen, hydroxy, nitro, cyano, amino, oxo and oxime or from a second group consisting of linear or branched (C₁-C₈)alkyl, (C₃-C₇)cycloalkyl, alkylcycloalkyl, perhaloalkyl, perhalocycloalkyl, aryl, aralkyl, alkylaryl, alkylheterocyclyl, aralkoxylalkyl, perhaloaryl, alkylheterocycloalkyl, heterocycloalkyl, perhaloheterocycloalkyl, heterocyclyl, perhaloheteroaryl, alkoxyalkyl, thioalkyl and thioaryl, wherein the substitutents from said second group are optionally substituted by halogen, hydroxy, nitro, cyano, amino, oxo, perhaloalkyl (C₁-C₆) and oxime and are optionally and independently bridged by —CO, —(CO)O—, —(CO)NH—, —NH—, —NR₁₄, -0-, —S—, —(SO)—, —(SO₂), —(SO₂)NH—, or —NH(CO)—;
R₁₂ and R₁₃ are independently selected from the group consisting of linear or branched (C₁-C₁₂)alkyl, (C₃-C₇)cycloalkyl, alkylcycloalkyl, aryl and heterocyclyl, wherein members of said group are optionally substituted by R₁₆;
R₁₄ and R₁₅ are independently selected from the group consisting of linear or branched (C₁-C₁₂)alkyl, (C₃-C₇)cycloalkyl, bicycloalkyl, aryl, and heterocyclyl wherein substituents of said group are optionally substituted by R₁₆;
R₁₆ is halogen, hydroxy, nitro, cyano, amino, oxo, perhaloalkyl (C₁-C₆), or oxime;
with the proviso that
(i) when R₁ is —C(O)R₃, then Z is —CH₂—C(O)-Rx; and
(ii) when Z is —CH₂—C(O)—R_{X} and R_{X} is OR₇, then R₇ is not hydrogen.

4. The compound as claimed in claim 3, wherein nitrogen containing ring of formula (I) having double bonds at C₂-C₃ and C₅-C₆.

5. The compound as claimed in claim 2, 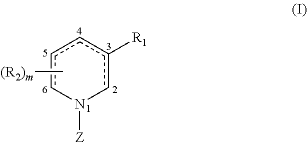
wherein, the dotted line in nitrogen containing ring represents:
(a) two double bonds at C₂-C₃ and C₅-C₆, or
(b) one double between either (i) at C₂-C₃ or (ii) at C₃-C₄ or (iii) at C₄-C₅, or
(c) absence of any double bonds;
R₁ is —COR₃;
Z is —CH₂—C(O)—R_{X};
R_{X} is R₇, OR₇, —N(R₇)(R₁₀), —N(R₇)N(R₇)(R₁₀) or CH(R₇)C(O)R₈;
R₂ is aryl and m is 0 or 1;
R₃ is —R₄—R₅ or —N(R₇)N(R₇)R₉;
R₄ is —N(R₇)R₆O—, —OR₆O— or —OR₆N(R₇), where R₆ is alkylene;
R₅ is hydrogen, alkyl, —COR₇ or COR₁₀;
R₇ is H, alkyl aryl or heterocyclyl;
R₈ is selected from R₇, OR₇ or NR₇R₁₀;
R₉ is selected from the group consisting of hydrogen, aryl, heterocyclyl, —C(O)R₁₀, —SO₂R₁₀ and C(O)NHR₁₀;
R₁₀ is selected from the group consisting of H, alkyl, alkoxy, aryl and heterocyclyl; with the proviso that;
when Z is —CH₂—C(0)-R_{X} and R_{X} is OR₇, then R₇ is not hydrogen.

6. The compound as claim in claim 2, which is selected from the group consisting of:
5-bromo-N′-(methylsulfonyl)-1-[2-oxo-2-(thiophen-2-yl)ethyl]-1,4-dihydropyridine-3-carbohydrazide;
ethyl 2-({1-[2-oxo-2-(thiophen-2-yl)ethyl]-1,4-dihydropyridin-3-yl}carbonyl)hydrazinecarboxylate;
2-[3-{5-[(3,5-dimethyl-1H-pyrazol-1-yl)methyl]-1H-pyrazol-3-yl}pyridin-1(4H)-yl]-1-(thiophen-2-yl)ethanone;
2-{3-[5-benzyl-1-(pyridin-2-yl)-1H-pyrazol-3-yl]pyridin-1(4H)-yl}-1-(thiophen-2-yl)ethanone;
1-[2-(5-chlorothiophen-2-yl)-2-oxoethyl]-N′-(methylsulfonyl)-1,4-dihydropyridine-3-carbohydrazide;
1-[2-(4-nitrothiophen-2-yl)-2-oxoethyl]-N′-({1-[2-(4-nitrothiophen-2-yl)-2-oxoethyl]-1,4-dihydropyridin-3-yl}carbonyl)-1,4-dihydropyridine-3-carbohydrazide;
6-methyl-N′-(methylsulfonyl)-1-[2-oxo-2-(thiophen-2-yl)ethyl]-1,4-dihydropyridine-3-carbohydrazide;
1-[2-(5-methylthiophen-2-yl)-2-oxoethyl]-N′-({1-[2-(5-methylthiophen-2-yl)-2-oxoethyl]-1,4-dihydropyridin-3-yl}carbonyl)-1,4-dihydropyridine-3-carbohydrazide;
diethyl 1,1′-{hydrazine-1,2-diylbis[carbonylpyridine-3,1(4H)-diyl(1-oxoethane-2,1-diyl)]}dipyrrolidine-2-carboxylate;
ethyl 3-{[3-{[2-(methylsulfonyl)hydrazinyl]carbonyl}pyridin-1(4H)-yl]acetyl}-1,3-thiazolidine-4-carboxylate;
1-[2-(5-chlorothiophen-2-yl)-2-oxoethyl]-N′-({1-[2-(5-chlorothiophen-2-yl)-2-oxoethyl]-1,4-dihydropyridin-3-yl}carbonyl)-1,4-dihydropyridine-3-carbohydrazide;
N′-(methylsulfonyl)-1-[2-(5-methylthiophen-2-yl)-2-oxoethyl]-1,4-dihydropyridine-3-carbohydrazide;
N′-(methylsulfonyl)-1-[2-(4-nitrothiophen-2-yl)-2-oxoethyl]-1,4-dihydropyridine-3-carbohydrazide;
N-phenyl-2-{3-[(2-phenylhydrazinyl)carbonyl]pyridin-1(4H)-yl}acetamide;
N′-(methylsulfonyl)-1-[2-(5-nitrothiophen-2-yl)-2-oxoethyl]-1,4-dihydropyridine-3-carbohydrazide;
1-[2-oxo-2-(thiophen-2-yl)ethyl]-N′-[(trifluoromethyl)sulfonyl]-1,4-dihydropyridine-3-carbohydrazide;
1-[2-oxo-2-(thiophen-2-yl)ethyl]-N′-phenyl-1,4-dihydropyridine-3-carbohydrazide;
N′-[(4-methoxyphenyl)sulfonyl]-1-[2-oxo-2-(thiophen-2-yl)ethyl]-1,4-dihydropyridine-3-carbohydrazide;
2-{[1-(2-oxo-2-phenylethyl)-1,4-dihydropyridin-3-yl]carbonyl}-N-phenylhydrazine carboxamide;
2-[3-{[2-(benzylsulfonyl)hydrazinyl]carbonyl}pyridin-1(4H)-yl]-N-phenylacetamide;
1-(2-oxo-2-phenylethyl)-N′-phenyl-1,4-dihydropyridine-3-carbohydrazide;
N′-[(4-methoxyphenyl)sulfonyl]-1-(2-oxo-2-phenylethyl)-1,4-dihydropyridine-3-carbohydrazide;
N-cyclopropyl-2-[3-{5-[(3,5-dimethyl-1H-pyrazol-1-yl)methyl]-1H-pyrazol-3-yl}pyridin-1(4H)-yl]acetamide;
2-[3-{5-[(3,5-dimethyl-1H-pyrazol-1-yl)methyl]-1H-pyrazol-3-yljpyridin-1(4H)-yl]-1-(5-nitrothiophen-2-yl)ethanone;
2-[3-{3-[(3,5-dimethyl-1H-pyrazol-1-yl)methyl]-1-(pyridin-2-yl)-1H-pyrazol-5-yl}pyridin-1(4H)-yl]-1-(thiophen-2-yl)ethanone;
2-[3-(5-benzyl-1H-pyrazol-3-yl)pyridin-1(4H)-yl]-N-cyclopropylacetamide;
2,2′-[1H-pyrazole-3,5-diylbis(pyridine-3,1(4H)-diyl)]bis[1-(thiophen-2-yl)ethanone];
2-[3-(5-benzyl-1-phenyl-1H-pyrazol-3-yl)pyridin-1(4H)-yl]-1-(thiophen-2-yl)ethanone;
2-[3-(5-benzyl-1H-pyrazol-3-yl)pyridin-1(4H)-yl]-1-(5-methylthiophen-2-yl)ethanone;
2-[3-{5-[(3,5-dimethyl-1H-pyrazol-1-yl)methyl]-1-phenyl-1H-pyrazol-3-yl}pyridin-1(4H)-yl]-1-(thiophen-2-yl)ethanone;
N′-({1-[2-oxo-2-(thiophen-2-yl)ethyl]-1,4-dihydropyridin-3-yl}carbonyl)pyridine-3-carbohydrazide;
2-[3-(5-benzyl-1H-pyrazol-3-yl)pyridin-1(4H)-yl]-1-phenylethanone;
2-[3-(5-benzyl-1-phenyl-1H-pyrazol-3-yl)pyridin-1(4H)-yl]-N-cyclopropylacetamide;
2-[3-{5-[(3,5-dimethyl-1H-pyrazol-1-yl)methyl]-1H-pyrazol-3-yl}pyridin-1(4H)-yl]-1-phenylethanone;
2-[3-{5-[(3,5-dimethyl-1H-pyrazol-1-yl)methyl]-1H-pyrazol-3-yl}pyridin-1(4H)-yl]-1-(5-methylthiophen-2-yl)ethanone;
2-[3-(5-benzyl-1-phenyl-1H-pyrazol-3-yl)pyridin-1(4H)-yl]-1-phenylethanone;
2-{3-[5-(2-cyclohexylethyl)-1H-pyrazol-3-yl]pyridin-1(4H)-yl}-1-(5-methylthiophen-2-yl)ethanone;
2-{3-[5-(2-cyclohexylethyl)-1H-pyrazol-3-yl]pyridin-1(4H)-yl}-N-cyclopropylacetamide;
2-{3-[5-(2-cyclohexylethyl)-1H-pyrazol-3-yl]pyridin-1(4H)-yl}-1-phenylethanone;
2-[3-(5-benzyl-1-cyclohexyl-1H-pyrazol-3-yl)pyridin-1(4H)-yl]-N-cyclopropylacetamide;
2-{3-[5-(phenoxymethyl)-1H-pyrazol-3-yl)pyridin-1(4H)-yl}-1-(thiophen-2-yl)ethanone;
2-[3-(5-benzyl-1H-pyrazol-3-yl)pyridin-1(4H)-yl]-N-(tricyclo[3.3.1.13,7]dec-1-yl)acetamide;
2-[3-{5-[(3,5-dimethyl-1H-pyrazol-1-yl)methyl]-1-phenyl-1H-pyrazol-3-yl}pyridin-1(4H)-yl]-1-phenylethanone;
2-[3-{1-cyclohexyl-5-[(3,5-dimethyl-1H-pyrazol-1-yl)methyl]-1H-pyrazol-3-yl}pyridin-1(4H)-yl]-1-(4-nitrothiophen-2-yl)ethanone;
2-{3-[3-(2-cyclohexylethyl)-1H-pyrazol-5-yl]pyridin-1(4H)-yl}-1-(4-nitrothiophen-2-yl)ethanone;
2-{3-[3-(phenoxymethyl)-1-phenyl-1H-pyrazol-5-yl]pyridin-1(4H)-yl}-1-(thiophen-2-yl)ethanone;
2-[3-(3-benzyl-1-phenyl-1H-pyrazol-5-yl)pyridin-1(4H)-yl]-1-(4-nitrothiophen-2-yl)ethanone;
N-cyclopropyl-2-{3-[3-(phenoxymethyl)-1H-pyrazol-5-yl]pyridin-1(4H)-yl}acetamide;
2-[3-{1-cyclohexyl-3-[(3,5-dimethyl-1H-pyrazol-1-yl)methyl]-1H-pyrazol-5-yl}pyridin-1(4H)-yl]-N-cyclopropylacetamide;
1-(5-chlorothiophen-2-yl)-2-{3-[3-(phenoxymethyl)-1H-pyrazol-5-yl]pyridin-1(4H)-yl}ethanone;
2-{3-[3-(phenoxymethyl)-1-phenyl-1H-pyrazol-5-yl]pyridin-1(4H)-yl}-1-phenylethanone;
2-[3-{1-cyclohexyl-3-[(3,5-dimethyl-1H-pyrazol-1-yl)methyl]-1H-pyrazol-5-yl}pyridin-1(4H)-yl]-1-(thiophen-2-yl)ethanone;
N-cyclopropyl-2-{3-[3-(phenoxymethyl)-1-phenyl-1H-pyrazol-5-yl]pyridin-1(4H)-yl}acetamide;
2-{3-[3-(2-cyclohexylethyl)-1-phenyl-1H-pyrazol-5-yl]pyridin-1(4H)-yl}-1-(thiophen-2-yl)ethanone;
2-{3-[1-cyclohexyl-3-(phenoxymethyl)-1H-pyrazol-5-yl]pyridin-1(4H)-yl}-1-(thiophen-2-yl)ethanone;
1-(naphthalen-2-yl)-2-{3-[3-(phenoxymethyl)-1H-pyrazol-5-yljpyridin-1(4H)-yl}ethanone;
1-benzyl-3-(3-benzyl-1H-pyrazol-5-yl)-1,4-dihydropyridine;
2-{3-[3-(naphthalen-1-ylmethyl)-1H-pyrazol-5-yl]pyridin-1(4H)-yl}-1-(thiophen-2-yl)ethanone;
1-phenyl-2-{3-[3-(thiophen-2-ylmethyl)-1H-pyrazol-5-yl]pyridin-1(4H)-yl}ethanone;
1-(5-methylthiophen-2-yl)-2-{3-[3-(2-phenylethyl)-1H-pyrazol-5-yl]pyridin-1(4H)-yl}ethanone;
1-(5-methylthiophen-2-yl)-2-{3-[3-(3-phenoxypropyl)-1H-pyrazol-5-yl]pyridin-1(4H)-yl}ethanone;
3-(3-benzyl-1H-pyrazol-5-yl)-1-(propan-2-yl)-1,4-dihydropyridine;
1-(5-methylthiophen-2-yl)-2-[3-{3-[(phenylsulfanyl)methyl]-1H-pyrazol-5-yl}pyridin-1(4H)-yl]ethanone;
N-(2-hydroxyethyl)-1-(2-oxo-2-phenylethyl)-1,4-dihydropyridine-3-carboxamide;
2-[3-{3-[(1-methyl-1H-indol-3-yl)methyl]-1H-pyrazol-5-yl}pyridin-1(4H)-yl]-1-(thiophen-2-yl)ethanone;
2-[3-(3-methyl-1H-pyrazol-5-yl)pyridin-1(4H)-yl]-1-(naphthalen-2-yl)ethanone;
2-[3-(3-benzyl-1H-pyrazol-5-yl)pyridin-1(4H)-yl]-N-(2,3-dihydro-1,4-benzodioxin-6-yl)acetamide;
2-[3-bromo-5-(3-phenyl-1H-pyrazol-5-yl)pyridin-1(4H)-yl]-1-(thiophen-2-yl)ethanone;
2-[3-(3-phenyl-1H-pyrazol-5-yl)pyridin-1(4H)-yl]-1-(thiophen-2-yl)ethanone;
1-[2-oxo-2-(thiophen-2-yl)ethyl]-1,4-dihydropyridine-3-carboxamide;
1-(2-oxo-2-phenylethyl)-N′-{[1-(2-oxo-2-phenylethyl)-1,4-dihydropyridin-3-yl]carbonyl}-1,4-dihydropyridine-3-carbohydrazide;
1-[2-(2,4-dichlorophenyl)-2-oxoethyl]-N′-({1-[2-(2,4-dichlorophenyl)-2-oxoethyl]-1,4-dihydropyridin-3-yl}carbonyl)-1,4-dihydropyridine-3-carbohydrazide;
N-(2-hydroxyethyl)-1-(2-oxopropyl)-1,4-dihydropyridine-3-carboxamide;
N-(2-hydroxyethyl)-1-[2-oxo-2-(thiophen-2-yl)ethyl]-1,4-dihydropyridine-3-carboxamide;
1-[2-oxo-2-(thiophen-2-yl)ethyl]-N′-({1-[2-oxo-2-(thiophen-2-yl)ethyl]-1,4-dihydropyridin-3-yl}carbonyl)-1,4-dihydropyridine-3-carbohydrazide;
N-(2-hydroxyethyl)-1-[2-oxo-2-(phenylamino)ethyl]-1,4-dihydropyridine-3-carboxamide;
1-(2-hydrazinyl-2-oxoethyl)-N-(2-hydroxyethyl)-1,4-dihydropyridine-3-carboxamide;
1-(2-hydrazinyl-2-oxoethyl)-1,4-dihydropyridine-3-carbohydrazide;
2-[({1-[2-(2,4-dichlorophenyl)-2-oxoethyl]-1,4-dihydropyridin-3-yl}carbonyl)amino]ethyl benzoate;
1-[2-oxo-2-(thiophen-2-yl)ethyl]-N′-(pyridin-2-yl)-1,4-dihydropyridine-3-carbohydrazide;
1-(2-oxo-2-phenylethyl)-N′-(pyridin-2-yl)-1,4-dihydropyridine-3-carbohydrazide;
1-(2-oxo-2-phenylethyl)-1,4-dihydropyridine-3-carbohydrazide;
N′-(methylsulfonyl)-1-[2-oxo-2-(thiophen-2-yl)ethyl]-1,4-dihydropyridine-3-carbohydrazide;
N′-(methylsulfonyl)-1-(2-oxo-2-phenylethyl)-1,4-dihydropyridine-3-carbohydrazide;
1-(2-oxo-2-phenylethyl)-N′-(phenylsulfonyl)-1,4-dihydropyridine-3-carbohydrazide;
6-chloro-1-(2-oxo-2-phenylethyl)-N′-(phenylsulfonyl)-1,4-dihydropyridine-3-carbohydrazide;
2-[(methoxycarbonyl)oxy]ethyl 1-(2-oxo-2-phenylethyl)-1,4-dihydropyridine-3-carboxylate;
2-methoxyethyl 1-[2-(2,4-dichlorophenyl)-2-oxoethyl]-1,4-dihydropyridine-3-carboxylate;
2-[({1-[2-oxo-2-(phenylamino)ethyl]-1,4-dihydropyridin-3-yl}carbonyl)amino]ethyl benzoate;
N-phenyl-2-[3-{[2-(phenylsulfonyl)hydrazinyl]carbonyl}pyridin-1(4H)-yl]acetamide;
2-[3-({2-[4-methylphenyl)sulfonyl]hydrazinyl}carbonyl)pyridin-1(4H)-yl]-N-phenylacetamide;
2-[(phenylcarbonyl)oxy]ethyl 1-(2-oxo-2-phenylethyl)-1,4-dihydropyridine-3-carboxylate;
1-[2-oxo-2-(thiophen-2-yl)ethyl]-N′-(phenylcarbonyl)-1,4-dihydropyridine-3-carbohydrazide;
N′-(benzylsulfonyl)-1-(2-oxo-2-phenylethyl)-1,4-dihydropyridine-3-carbohydrazide;
N-(2-hydroxyethyl)-1-[2-oxo-2-(phenylamino)ethyl]-1,4-dihydropyridine-3-carboxamide;
N′-(3-cyclohexylpropanoyl)-1-[2-oxo-2-(thiophen-2-yl)ethyl]-1,4-dihydropyridine-3-carbohydrazide;
2-[3-{[2-(3-cyclohexylpropanoyl)hydrazinyl]carbonyl}pyridin-1(4H)-yl]-N-phenylacetamide;
2-[({1-[2-oxo-2-(thiophen-2-yl)ethyl]-1,4-dihydropyridin-3-yl}carbonyl)amino]ethyl benzoate;
2-({[1-(4-ethoxy-2,4-dioxobutyl)-1,4-dihydropyridin-3-yl]carbonyl}amino)ethyl benzoate;
1-[2-(furan-2-yl)-2-oxoethyl]-N′-({1-[2-(furan-2-yl)-2-oxoethyl]-1,4-dihydropyridin-3-yl}carbonyl)-1,4-dihydropyridine-3-carbohydrazide;
1-[2-(2,5-dichlorophenyl)-2-oxoethyl]-N-(2-methoxyethyl)-1,4-dihydropyridine-3-carboxamide;
2,2′-[hydrazine-1,2-diylbis(carbonylpyridine-3,1(4H)-diyl)]bis(N-cyclopropylacetamide);
1-[2-(cyclopropylamino)-2-oxoethyl]-N-(2-methoxyethyl)-1,4-dihydropyridine-3-carboxamide;
2-chloro-N′-({1-[2-oxo-2-(thiophen-2-yl)ethyl]-1,4-dihydropyridin-3-yl}carbonyl)pyridine-3-carbohydrazide;
2-[3-{[2-(methylsulfonyl)hydrazinyl]carbonyl}pyridin-1(4H)-yl]-N-(propan-2-yl)acetamide;
N′-(methylsulfonyl)-1-[2-oxo-2-(pyrrolidin-1-yl)ethyl]-1,4-dihydropyridine-3-carbohydrazide;
5-bromo-N-(2-methoxyethyl)-1-[2-oxo-2-(thiophen-2-yl)ethyl]-1,4-dihydropyridine-3-carboxamide;
methyl 5-{[2-({1-[2-oxo-2-(thiophen-2-yl)ethyl]-1,4-dihydropyridin-3-yl}carbonyl)hydrazinyl]carbonyl}pyridine-2-carboxylate;
2-[3-(3-benzyl-1H-pyrazol-5-yl)pyridin-1(4H)-yl]-1-(thiophen-2-yl)ethanone;
6-methyl-1-[2-oxo-2-(thiophen-2-yl)ethyl]-N′-({1-[2-oxo-2-(thiophen-2-yl)ethyl]-1,4-dihydropyridin-3-yl}carbonyl)-1,4-dihydropyridine-3-carbohydrazide;
1-[2-oxo-2-(thiophen-2-yl)ethyl]-N′-(propan-2-ylsulfonyl)-1,4-dihydropyridine-3-carbohydrazide;
2-[3-(5-benzyl-1,2-oxazol-3-yl)pyridin-1(4H)-yl]-1-(thiophen-2-yl)ethanone;
1-[2-(4-benzylpiperidin-1-yl)-2-oxoethyl]-N′-(methylsulfonyl)-1,4-dihydropyridine-3-carbohydrazide;
ethyl 1-{[3-{[2-(methylsulfonyl)hydrazinyl]carbonyl}pyridin-1(4H)-yl]acetyl}prolinate;
6-hydroxy-N′-(methylsulfonyl)-1-[2-oxo-2-(thiophen-2-yl)ethyl]-1,4,5,6-tetrahydropyridine-3-carbohydrazide;
2,6-dihydroxy-N′-(methylsulfonyl)-1-[2-oxo-2-(thiophen-2-yl)ethyl]piperidine-3-carbohydrazide;
N′-(methylsulfonyl)-6-oxo-1-[2-oxo-2-(thiophen-2-yl)ethyl]-1,6-dihydropyridine-3-carbohydrazide;
1-[2-(4-bromophenyl)-2-oxoethyl]-N′-(methylsulfonyl)-1,4-dihydropyridine-3-carbohydrazide;
1-[2-(4-methoxyphenyl)-2-oxoethyl]-N′-(methylsulfonyl)-1,4-dihydropyridine-3-carbohydrazide;
2-[3-({2-[4-methylphenyl)sulfonyl]hydrazinyl}carbonyl)pyridin-1(4H)-yl]-N-phenylacetamide;
2-[3-({2-[(4-tert-butylphenyl)sulfonyl]hydrazinyl}carbonyl)pyridin-1(4H)-yl]-N-phenylacetamide;
N′-[(4-methylphenyl)sulfonyl]-1-[2-oxo-2-(thiophen-2-yl)ethyl]-1,4-dihydropyridine-3-carbohydrazide;
1-[2-(4-bromophenyl)-2-oxoethyl]-N-(2-hydroxyethyl)-1,4-dihydropyridine-3-carboxamide;
1-[2-oxo-2-(thiophen-2-yl)ethyl]-N′-(phenylsulfonyl)-1,4-dihydropyridine-3-carbohydrazide;
N′-[(4-tert-butylphenyl)sulfonyl]-1-[2-oxo-2-(thiophen-2-yl)ethyl]-1,4-dihydropyridine-3-carbohydrazide;
5-(4-methoxyphenyl)-N′-(methylsulfonyl)-1-[2-oxo-2-(thiophen-2-yl)ethyl]-1,4-dihydropyridine-3-carbohydrazide;
N′-[(4-methylphenyl)sulfonyl]-1-(2-oxo-2-phenylethyl)-1,4-dihydropyridine-3-carbohydrazide;
1-[2-oxo-2-(thiophen-2-yl)ethyl]-N′-(thiophen-2-ylcarbonyl)-1,4-dihydropyridine-3-arbohydrazide;
1-[2-(4-methoxyphenyl)-2-oxoethyl]-N′-[(4-methylphenyl)sulfonyl]-1,4-dihydropyridine-3-carbohydrazide;
2-{3-[3-(4-bromophenyl)-1,2-oxazol-5-yl]pyridin-1(4H)-yl}-1-(thiophen-2-yl)ethanone;
2-{3-[3-(4-methylphenyl)-1,2-oxazol-5-yl]pyridin-1(4H)-yl}-N-phenylacetamide;
N′-[(4-methylphenyl)sulfonyl]-1-[2-oxo-2-(thiophen-2-yl)ethyl]-1,4,5,6-tetrahydropyridine-3-carbohydrazide;
N-(2-hydroxyethyl)-1-[2-oxo-2-(thiophen-2-yl)ethyl]-1,4,5,6-tetrahydropyridine-3-carboxamide;
2-[3-({2-[4-methylphenyl)sulfonyl]hydrazinyl}carbonyl)piperidin-1-yl]-N-phenylacetamide;
1-[2-oxo-2-(thiophen-2-yl)ethyl]-N′-(phenylsulfonyl)piperidine-3-carbohydrazide;
N′-[(4-methylphenyl)sulfonyl]-1-(2-oxo-2-phenylethyl)piperidine-3-carbohydrazide;
1-[2-(4-methoxyphenyl)-2-oxoethyl]-N′-[(4-methylphenyl)sulfonyl]piperidine-3-carbohydrazide;
1-[2-(4-methoxyphenyl)-2-oxoethyl]-N′-(methylsulfonyl)piperidine-3-carbohydrazide;
N′-(methylsulfonyl)-1-[2-(5-methylthiophen-2-yl)-2-oxoethyl]piperidine-3-carbohydrazide;
N′-[(4-tert-butylphenyl)sulfonyl]-1-[2-oxo-2-(thiophen-2-yl)ethyl]piperidine-3-carbohydrazide;
methyl{3-[5-(4-bromophenyl)-1H-pyrazol-3-yl]piperidin-1-yl}acetate;
1-[2-(4-bromophenyl)-2-oxoethyl]-N′-(methylsulfonyl)-1,2,5,6-tetrahydropyridine-3-carbohydrazide;
1-[2-(2,4-dichlorophenyl)-2-oxoethyl]-N′-(methylsulfonyl)-1,4-dihydropyridine-3-carbohydrazide;
1-[2-oxo-2-(thiophen-2-yl)ethyl]-N′-(phenylsulfonyl)-1,2,5,6-tetrahydropyridine-3-carbohydrazide;
N′-[(2Z)-2-(ethenylsulfanyl)but-2-enoyl]-1-[2-oxo-2-(thiophen-2-yl)ethyl]-1,2,5,6-tetrahydropyridine-3-carbohydrazide;
N-(4-chlorophenyl)-2-({1-[2-oxo-2-(thiophen-2-yl)ethyl]-1,2,5,6-tetrahydropyridin-3-yl}carbonyl)hydrazinecarboxamide;
1-[2-(4-methoxyphenyl)-2-oxoethyl]-N′-(methylsulfonyl)-1,2,5,6-tetrahydropyridine-3-carbohydrazide;
1-[2-(4-methoxyphenyl)-2-oxoethyl]-N′-[(4-methylphenyl)sulfonyl]-1,2,5,6-tetrahydropyridine-3-carbohydrazide;
N′-(methylsulfonyl)-1-[2-(5-methylthiophen-2-yl)-2-oxoethyl]-1,2,5,6-tetrahydropyridine-3-carbohydrazide;
N′-[(4-tert-butylphenyl)sulfonyl]-1-[2-oxo-2-(thiophen-2-yl)ethyl]-1,2,5,6-tetrahydropyridine-3-carbohydrazide;
2-{5-[3-(4-methoxyphenyl)-1,2-oxazol-5-yl]-3,6-dihydropyridin-1(2H)-yl}-1-(thiophen-2-yl)ethanone;
methyl{5-[5-(4-bromophenyl)-1H-pyrazol-3-yl]-3,6-dihydropyridin-1(2H)-yl}acetate;
2-{5-[3-(3-hydroxyphenyl)-1,2-oxazol-5-yl]-3,6-dihydropyridin-1(2H)-yl}-1-(thiophen-2-yl)ethanone;
2-{5-[5-(4-hydroxyphenyl)-1H-pyrazol-3-yl]-3,6-dihydropyridin-1(2H)-yl}acetamide;
2-{5-[5-(4-fluorophenyl)-1H-pyrazol-3-yl]-3,6-dihydropyridin-1(2H)-yl}acetamide;
1-[2-(4-methoxyphenyl)-2-oxoethyl]-N′-(methylsulfonyl)-1,2,3,6-tetrahydropyridine-3-carbohydrazide;
N′-(methylsulfonyl)-1-[2-(5-methylthiophen-2-yl)-2-oxoethyl]-1,2,3,6-tetrahydropyridine-3-carbohydrazide;
N′-(methylsulfonyl)-1-[2-oxo-2-(thiophen-2-yl)ethyl]-1,4,5,6-tetrahydropyridine-3-carbohydrazide;
N′-(methylsulfonyl)-1-[2-oxo-2-(thiophen-2-yl)ethyl]piperidine-3-carbohydrazide;
N′-(methylsulfonyl)-1-[2-oxo-2-(thiophen-2-yl)ethyl]-1,2,5,6-tetrahydropyridine-3-carbohydrazide;
N′-(methylsulfonyl)-1-[2-oxo-2-(thiophen-2-yl)ethyl]-1,2,3,6-tetrahydropyridine-3-carbohydrazide;
N′-(methylsulfonyl)-1-[2-oxo-2-(thiophen-2-yl)ethyl]-1,4,5,6-tetrahydropyridine-3-carbohydrazide;
2-{3-[3-(3-hydroxyphenyl)-1,2-oxazol-5-yl]pyridin-1(4H)-yl}-1-(thiophen-2-yl)ethanone;
2-{3-[5-(4-hydroxyphenyl)-1H-pyrazol-3-yl]pyridin-1(4H)-yl}acetamide;
1-[2-(1-benzofuran-2-yl)-2-oxoethyl]-N′-(methylsulfonyl)-1,2,5,6-tetrahydropyridine-3-carbohydrazide;
ethyl{5-[3-(4-fluorophenyl)-1H-pyrazol-5-yl]-3,6-dihydropyridin-1(2H)-yl}acetate;
2-{5-[3-(4-bromophenyl)-1H-pyrazol-5-yl]-3,6-dihydropyridin-1(2H)-yl}acetamide;
1-[2-(1-benzofuran-2-yl)-2-oxoethyl]-N′-(methylsulfonyl)piperidine-3-carbohydrazide;
methyl 1-{[3-{[2-(methylsulfonyl)hydrazinyl]carbonyl}pyridin-1(4H)-yl]acetyl}prolinate;
5-methyl-N′-(methylsulfonyl)-1-[2-oxo-2-(thiophen-2-yl)ethyl]-1,4-dihydropyridine-3-carbohydrazide;
6-methoxy-N′-(methylsulfonyl)-1-[2-oxo-2-(thiophen-2-yl)ethyl]-1,4-dihydropyridine-3-carbohydrazide;
N′-(methylsulfonyl)-1-[2-oxo-2-(thiophen-2-yl)ethyl]-6-(pyrrolidin-1-yl)-1,4-dihydropyridine-3-carbohydrazide;
6-chloro-N′-(methylsulfonyl)-1-[2-oxo-2-(thiophen-2-yl)ethyl]-1,4-dihydropyridine-3-carbohydrazide;
2-methyl-N′-(methylsulfonyl)-1-[2-oxo-2-(thiophen-2-yl)ethyl]-1,4-dihydropyridine-3-carbohydrazide;
2-(methylsulfanyl)-N′-(methylsulfonyl)-1-[2-oxo-2-(thiophen-2-yl)ethyl]-1,4-dihydropyridine-3-carbohydrazide;
2-(dimethylamino)ethyl 1-[2-oxo-2-(thiophen-2-yl)ethyl]-1,4-dihydropyridine-3-carboxylate;
2-ethoxyethyl 1-[2-oxo-2-(thiophen-2-yl)ethyl]-1,4-dihydropyridine-3-carboxylate;
4-(5-{1-[2-oxo-2-(phenylamino)ethyl]-1,4-dihydropyridin-3-yl}-1H-pyrazol-3-yl)phenyl;
4-(5-{1-[2-oxo-2-(phenylamino)ethyl]-1,4-dihydropyridin-3-yl}-1-phenyl-1H-pyrazol-3-yl)phenyl;
2-[({1-[2-oxo-2-(phenylamino)ethyl]-1,4-dihydropyridin-3-yl}carbonyl)amino]ethyl 4-methoxybenzoate;
6-methoxy-N′-(methylsulfonyl)-1-[2-oxo-2-(thiophen-2-yl)ethyl]-1,4,5,6-tetrahydro pyridine-3-carbohydrazide and pharmaceutically acceptable salts thereof.

7. The compound as claimed in claim 5, which is selected from the group consisting of:
1-[2-(5-chlorothiophen-2-yl)-2-oxoethyl]-N′-(methylsulfonyl)-1,4-dihydropyridine-3-carbohydrazide;
N′-(methylsulfonyl)-1-[2-(5-methylthiophen-2-yl)-2-oxoethyl]-1,4-dihydropyridine-3-carbohydrazide;
N′-(methylsulfonyl)-1-[2-oxo-2-(thiophen-2-yl)ethyl]-1,4-dihydropyridine-3-carbohydrazide;
2-[({1-[2-oxo-2-(phenylamino)ethyl]-1,4-dihydropyridin-3-yl}carbonyl)amino]ethyl benzoate;
1-[2-(4-bromophenyl)-2-oxoethyl]-N′-(methylsulfonyl)-1,4-dihydropyridine-3-carbohydrazide;
1-[2-(4-methoxyphenyl)-2-oxoethyl]-N′-(methylsulfonyl)-1,4-dihydropyridine-3-carbohydrazide;
2-[3-({2-[4-methylphenyl)sulfonyl]hydrazinyl}carbonyl)pyridin-1(4H)-yl]-N-phenylacetamide;
N′-[(4-methylphenyl)sulfonyl]-1-[2-oxo-2-(thiophen-2-yl)ethyl]-1,4-dihydropyridine-3-carbohydrazide;
1-[2-(4-bromophenyl)-2-oxoethyl]-N-(2-hydroxyethyl)-1,4-dihydropyridine-3-carboxamide;
5-(4-methoxyphenyl)-N′-(methylsulfonyl)-1-[2-oxo-2-(thiophen-2-yl)ethyl]-1,4-dihydropyridine-3-carbohydrazide;
N-(2-hydroxyethyl)-1-[2-oxo-2-(thiophen-2-yl)ethyl]-1,4,5,6-tetrahydropyridine-3-carboxamide;
1-[2-oxo-2-(thiophen-2-yl)ethyl]-N′-(phenylsulfonyl)piperidine-3-carbohydrazide;
1-[2-(4-methoxyphenyl)-2-oxoethyl]-N′-[(4-methylphenyl)sulfonyl]piperidine-3-carbohydrazide;
1-[2-(4-methoxyphenyl)-2-oxoethyl]-N′-(methylsulfonyl)piperidine-3-carbohydrazide;
N′-(methylsulfonyl)-1-[2-(5-methylthiophen-2-yl)-2-oxoethyl]piperidine-3-carbohydrazide;
N′-[(4-tert-butylphenyl)sulfonyl]-1-[2-oxo-2-(thiophen-2-yl)ethyl]piperidine-3-carbohydrazide;
1-[2-(4-bromophenyl)-2-oxoethyl]-N′-(methylsulfonyl)-1,2,5,6-tetrahydropyridine-3-carbohydrazide;
1-[2-(2,4-dichlorophenyl)-2-oxoethyl]-N′-(methylsulfonyl)-1,4-dihydropyridine-3-carbohydrazide;
1-[2-oxo-2-(thiophen-2-yl)ethyl]-N′-(phenylsulfonyl)-1,2,5,6-tetrahydropyridine-3-carbohydrazide;
N-(4-chlorophenyl)-2-({1-[2-oxo-2-(thiophen-2-yl)ethyl]-1,2,5,6-tetrahydropyridin-3-yl}carbonyl)hydrazinecarboxamide;
1-[2-(4-methoxyphenyl)-2-oxoethyl]-N′-(methylsulfonyl)-1,2,5,6-tetrahydropyridine-3-carbohydrazide;
1-[2-(4-methoxyphenyl)-2-oxoethyl]-N′-[(4-methylphenyl)sulfonyl]-1,2,5,6-tetrahydropyridine-3-carbohydrazide;
N′-(methylsulfonyl)-1-[2-(5-methylthiophen-2-yl)-2-oxoethyl]-1,2,5,6-tetrahydropyridine-3-carbohydrazide;
1-[2-(4-methoxyphenyl)-2-oxoethyl]-N′-(methylsulfonyl)-1,2,3,6-tetrahydropyridine-3-carbohydrazide;
N′-(methylsulfonyl)-1-[2-(5-methylthiophen-2-yl)-2-oxoethyl]-1,2,3,6-tetrahydropyridine-3-carbohydrazide;
N′-(methylsulfonyl)-1-[2-oxo-2-(thiophen-2-yl)ethyl]-1,4,5,6-tetrahydropyridine-3-carbohydrazide;
N′-(methylsulfonyl)-1-[2-oxo-2-(thiophen-2-yl)ethyl]piperidine-3-carbohydrazide;
N′-(methylsulfonyl)-1-[2-oxo-2-(thiophen-2-yl)ethyl]-1,2,5,6-tetrahydropyridine-3-carbohydrazide;
N′-(methylsulfonyl)-1-[2-oxo-2-(thiophen-2-yl)ethyl]-1,2,3,6-tetrahydropyridine-3-carbohydrazide;
N′-(methylsulfonyl)-1-[2-oxo-2-(thiophen-2-yl)ethyl]-1,4,5,6-tetrahydropyridine-3-carbohydrazide and pharmaceutically acceptable salts thereof.

8. A pharmaceutical composition comprising a compound as claimed in claim 2 and one or more pharmaceutically acceptable excipients.

9. A pharmaceutical composition comprising a compound as claimed in claim 1 and one or more pharmaceutically acceptable excipients.

10. The pharmaceutical composition as claimed in claim 8, wherein the pharmaceutical composition is an acid resistant composition.

11. A method of treating a neuropathy, nephropathy, microangiopathy, hypertension, heart failure, retinopathy, atherosclerosis, Alzheimer's diseases & dermatological disorders comprising administration of a therapeutically effective amount of a compound as claimed in claim 2.

12. The method of claim 11, which is a method to mitigate a pre-existing disease state, acute or chronic, or a recurring condition.

13. A method of treating a neuropathy, nephropathy, microangiopathy, hypertension, heart failure, retinopathy, atherosclerosis, Alzheimer's diseases & dermatological disorders comprising administration of therapeutically effective amount of a compound as claimed in claim 1.

14. Use of a compound as claimed in claim 2 for the manufacture of medicament for treatment of neuropathy, nephropathy, microangiopathy, hypertension, heart failure, retinopathy, atherosclerosis, Alzheimer's disease & dermatological disorders.

15. Use of a compound as claimed in claim 1 for the manufacture of medicament for treatment of neuropathy, nephropathy, microangiopathy, hypertension, heart failure, retinopathy, atherosclerosis, Alzheimer's disease & dermatological disorders.

16. (canceled)

17. The pharmaceutical composition as claimed in claim 9, wherein the pharmaceutical composition is an acid resistant composition.
